(19) United States
(12) Patent Application Publication
(10) Pub. No.: US 2024/0376089 A1
(43) Pub. Date: Nov. 14, 2024

(54) 3-HYDROXY-5-(ISOXAZOL-5-YL) PYRIDINE FORMYLGLYCINE COMPOUNDS, PREPARATION METHOD, PHARMACEUTICAL COMPOSITION AND USE

(71) Applicant: CHINA PHARMACEUTICAL UNIVERSITY, Jiangsu (CN)

(72) Inventors: Xiaojin ZHANG, Jiangsu (CN); Yue WU, Jiangsu (CN); Yafen CHEN, Jiangsu (CN); Zhihong LI, Jiangsu (CN); Linjian ZHANG, Jiangsu (CN); Xiang LI, Jiangsu (CN)

(73) Assignee: CHINA PHARMACEUTICAL UNIVERSITY, Jiangsu (CN)

(21) Appl. No.: 18/776,136

(22) Filed: Jul. 17, 2024

Related U.S. Application Data
(63) Continuation of application No. PCT/CN2022/078207, filed on Feb. 28, 2022.

(30) Foreign Application Priority Data
Jan. 25, 2022 (CN) 202210086106.2

(51) Int. Cl. C07D 413/04; A61K 31/4439; A61P 3/06

(52) U.S. Cl. CPC C07D 413/04; A61K 31/4439; A61P 3/06

(57) ABSTRACT

A series of 3-hydroxy-5-(isoxazol-5-yl) pyridine formylglycine compounds, a preparation method, a pharmaceutical composition, and the use. A structure of the compounds is shown as formula (I), and the compound derivatives comprise pharmaceutically acceptable salt thereof. The compounds and the pharmaceutical composition thereof have a high inhibition effect on HIF inhibition factors, and the activity can optimally reach the nano-molar concentration level, so that the compounds can be used for preparing a drug for treating fat metabolic diseases.

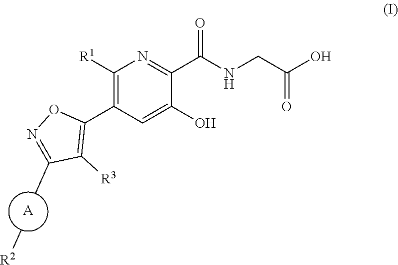

### CROSS-REFERENCE TO RELATED APPLICATION

This application is a continuation application of the international PCT application serial no. PCT/CN2022/078207, filed on Feb. 28, 2022, which claims the priority benefit of China application no. 202210086106.2, filed on Jan. 25, 2022. The entirety of each of the above-mentioned patent applications is hereby incorporated by reference herein and made a part of this specification.

### TECHNICAL FIELD

The present invention relates to a class of 3-hydroxy-5-(isoxazol-5-yl)picolinoyl glycine compounds, a preparation method, a pharmaceutical composition, and use, particularly to a class of 3-hydroxy-5-(isoxazol-5-yl)picolinoyl glycine compounds which can be prepared into a drug for treating a lipid metabolic disease, a preparation method, a pharmaceutical composition, and use.

### RELATED ART

Nonalcoholic steatohepatitis (NASH), also known as metabolic-dysfunction-associated fatty liver disease (MAFLD), has a global prevalence of up to 10%, seriously harms human health, and imposes a huge economic burden on society. NASH is an inflammatory subtype of nonalcoholic fatty liver disease (NAFLD) and is associated with disease progression, development of cirrhosis, and the need for liver transplantation. NASH is closely associated with obesity, dyslipidemia, type II diabetes, and metabolic syndrome. Currently, the FDA has not approved drugs for the treatment of NASH. It is estimated that 20% of patients with NASH will develop cirrhosis, and the mortality for patients with NASH is also significantly higher than that of the general population or patients with a non-inflammatory subtype of NAFLD.

The pathogenesis of NASH is extremely complex and remains unclear to date. Although lifestyle changes, including weight loss, have shown significant benefits for improving NASH, long-term maintenance still requires therapeutic intervention of drugs. However, there are currently no drugs approved for the treatment of NASH. Despite these challenges, the prospects for the development of NASH drugs are relatively broad and diversified, and more than 20 target-related drugs in the fields of metabolism, inflammation, and fibrosis are under development, those under phase III clinical studies including FXR agonists, PPAR-α/δ agonists, ASK1 inhibitors, THR-β agonists, etc. It is speculated that the global market size for NASH drugs will reach $40 billion in 2025, and the clinical needs are far from being met.

Factors inhibiting hypoxia-inducible factor (factors inhibiting HIF, FIHs) are a class of asparaginyl hydroxylase enzymes with a JmjC domain, which are well known as “cellular oxygen sensors”. In the presence of ferrous ions, oxygen, and 2OG, the specific asparagine residue of the HIF-α C-terminal transactivation domain (CTAD) can be hydroxylated, so that the binding capacity of HIF-α to a transcriptional enhancer p300/CBP is greatly reduced, thereby negatively regulating the transcriptional activity of HIF. Studies have shown that FIH gene knockout or silencing can cause a significant change in intracellular metabolism, and the decrease in FIH is accompanied by an increase in oxidative metabolism, which means that the inhibition of FIH can provide a new means for treating metabolic diseases. The deficiency of FIH can significantly reduce body weight and improve obesity. This is because the deficiency of FIH leads to an increase in oxygen consumption and calorie consumption of about 20%, as well as a significant increase in the metabolic rate. At the same time, the deficiency of FIH also leads to a significant reduction in adipocytes. The deficiency of FIH has a significant improvement and protective effect in mice with fatty liver induced by high-fat diet. FIH-deficient mice can show significantly lower triglyceride and cholesterol levels, as well as a significant improvement in liver hypertrophy. Therefore, the deficiency of FIH can improve lipid metabolic diseases.

For NASH, the existing treatment schemes are conservative treatment, and only can delay the disease progression. The pathogenesis of NASH is complex, and the course of the disease is long. There are more than twenty target drugs under clinical development, but no drugs have been approved by the FDA.

### SUMMARY OF INVENTION

Objective: aiming at the shortage of clinical therapeutic drugs for lipid metabolic diseases, the present invention aims to provide a class of 3-hydroxy-5-(isoxazol-5-yl)picolinoyl glycine compounds which have a brand-new mechanism and can effectively treat lipid metabolic diseases such as hyperlipidemia, NASH, etc., a preparation method, a pharmaceutical composition, and use.

Technical scheme: As a first aspect to which the present invention relates, the 3-hydroxy-5-(isoxazol-5-yl)picolinoyl glycine compound of the present invention has a structure of formula (I), and the compound comprises a pharmaceutically acceptable salt thereof:

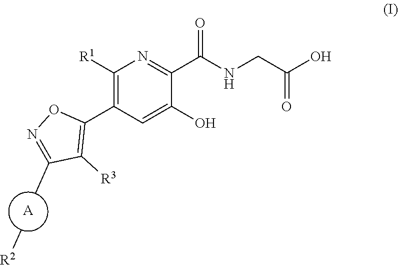

wherein:
A represents an aromatic ring or an aliphatic ring;
R¹ represents hydrogen, halogen, or methyl;
R² represents one or more of hydrogen, C₁-C₄ alkyl, C₁-C₄ alkoxy, C₁-C₄ haloalkyl, halogen, cyano, or phenyl;
R³ represents hydrogen, C₁-C₆ alkyl, C₁-C₆ cycloalkyl, or an aromatic ring.

Preferably, in the structure described above:

A represents a benzene ring, a naphthalene ring, a 5- to 6-membered aromatic heterocyclic ring, cyclohexane, or cyclopropane.

It is found in the present invention that FIH small molecule inhibitors can improve lipid metabolic diseases such as hyperlipidemia, obesity, NASH, etc. by accelerating the mechanism of lipid metabolism. No FIH small molecule inhibitors have been reported before, and the compounds of the present invention are first-in-class FIH small molecule inhibitors and are expected to become therapeutic drugs for lipid metabolic diseases with a brand-new mechanism, opening up a new field for the research and development of drugs for lipid metabolic diseases, particularly NASH.

Preferably, in the structure described above:

R² represents one or more of hydrogen, methyl, tert-butyl, methoxy, trifluoromethyl, fluoro, chloro, bromo, cyano, or phenyl.

Preferably, in the structure described above:

R³ represents hydrogen, methyl, tert-butyl, cyclopropyl, cyclohexyl, or phenyl.

More specifically, the 3-hydroxy-5-(isoxazol-5-yl)picolinoyl glycine compound is selected from any one of the following compounds:

| Code | Chemical name | Structural formula |
|---|---|---|
| I-1 | 3-Hydroxy-5- (3-phenylisoxazol-5- yl)picolinoyl glycine | 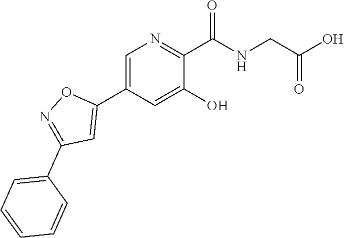 [SMILES: O=C(O)CNC(=O)c1ncc(-c2cc(-c3ccccc3)no2)cc1O] |
| I-2 | 3-Hydroxy-5- (3-m-tolylisoxazol-5- yl)picolinoyl glycine | 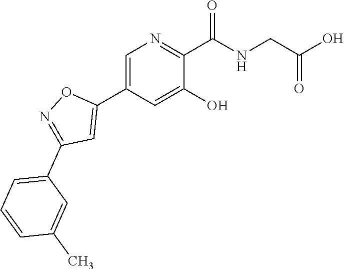 [SMILES: Cc1cccc(-c2cc(-c3cnc(C(=O)NCC(=O)O)c(O)c3)on2)c1] |
| I-3 | 3-Hydroxy-5- (3-biphenylisoxazol-5- yl)picolinoyl glycine | 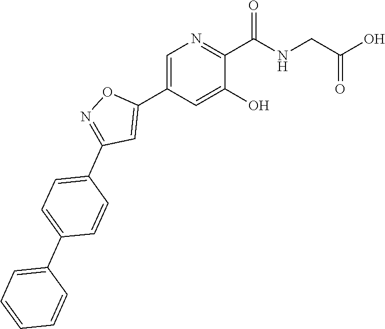 [SMILES: O=C(O)CNC(=O)c1ncc(-c2cc(-c3ccc(-c4ccccc4)cc3)no2)cc1O] |
| I-4 | 3-Hydroxy-5-(3-m- methoxyphenylisoxazol- 5-yl)picolinoyl glycine | 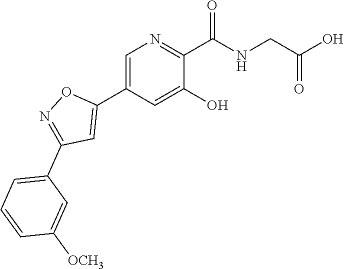 [SMILES: COc1cccc(-c2cc(-c3cnc(C(=O)NCC(=O)O)c(O)c3)on2)c1] |
| I-5 | 3-Hydroxy-5-(3-m- chlorophenylisoxazol- 5-yl)picolinoyl glycine | 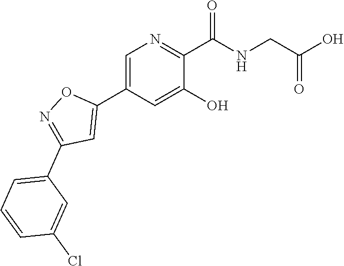 [SMILES: O=C(O)CNC(=O)c1ncc(-c2cc(-c3cccc(Cl)c3)no2)cc1O] |
| I-6 | 3-Hydroxy-5-(3-m- bromophenylisoxazol- 5-yl)picolinoyl glycine | 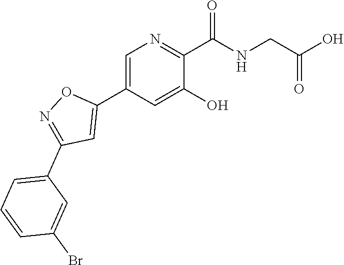 [SMILES: O=C(O)CNC(=O)c1ncc(-c2cc(-c3cccc(Br)c3)no2)cc1O] |
| I-7 | 3-Hydroxy-5-(3- cyclohexylisoxazol-5- yl)picolinoyl glycine | 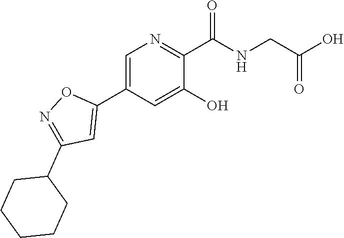 [SMILES: O=C(O)CNC(=O)c1ncc(-c2cc(C3CCCCC3)no2)cc1O] |
| I-8 | 3-Hydroxy-5-(3- cyclopropylisoxazol-5- yl)picolinoyl glycine | 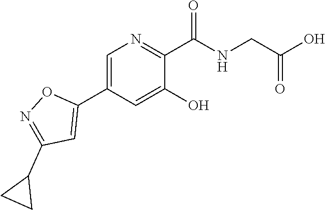 [SMILES: O=C(O)CNC(=O)c1ncc(-c2cc(C3CC3)no2)cc1O] |
| I-9 | 3-Hydroxy-5-(3-m- cyanophenylisoxazol- 5-yl)picolinoyl glycine | 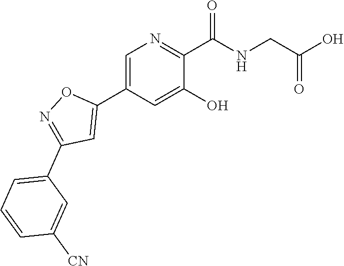 [SMILES: N#Cc1cccc(-c2cc(-c3cnc(C(=O)NCC(=O)O)c(O)c3)on2)c1] |
| I-10 | 3-Hydroxy-5-(3-m- trifluoromethylphenylisoxazol-5- yl)picolinoyl glycine | 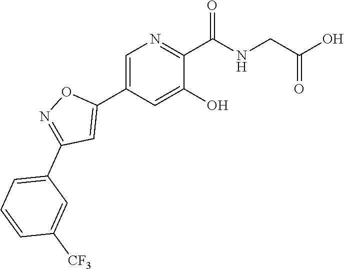 [SMILES: O=C(O)CNC(=O)c1ncc(-c2cc(-c3cccc(C(F)(F)F)c3)no2)cc1O] |
| I-11 | 3-Hydroxy-5-(3- (naphthyl-1-yl)isoxazol- 5-yl)picolinoyl glycine | 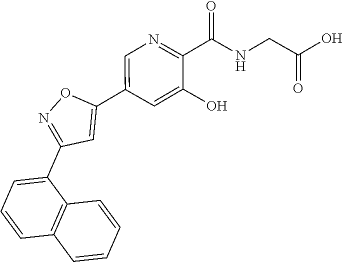 [SMILES: O=C(O)CNC(=O)c1ncc(-c2cc(-c3cccc4ccccc34)no2)cc1O] |
| I-12 | 3-Hydroxy-5-(3- (naphthyl-2-yl)isoxazol- 5-yl)picolinoyl glycine | 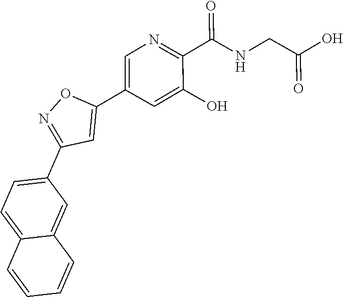 [SMILES: O=C(O)CNC(=O)c1ncc(-c2cc(-c3ccc4ccccc4c3)no2)cc1O] |
| I-13 | 3-Hydroxy-5- (3-o-tolylisoxazol- 5-yl)picolinoyl glycine | 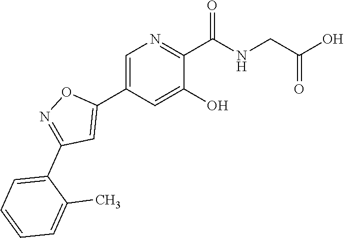 [SMILES: Cc1ccccc1-c1cc(-c2cnc(C(=O)NCC(=O)O)c(O)c2)on1] |
| I-14 | 3-Hydroxy-5- (3-p-tolylisoxazol- 5-yl)picolinoyl glycine | 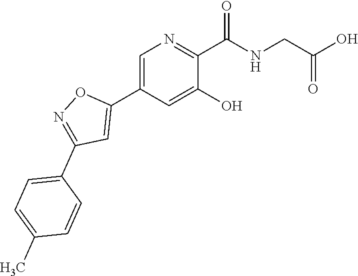 [SMILES: Cc1ccc(-c2cc(-c3cnc(C(=O)NCC(=O)O)c(O)c3)on2)cc1] |
| I-15 | 3-Hydroxy-5-(3-m- dimethylphenylisoxazol- 5-yl)picolinoyl glycine | 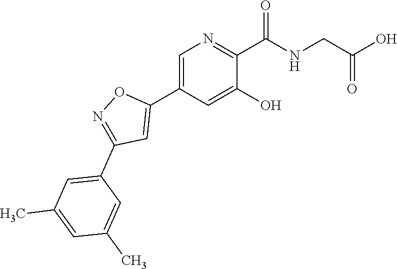 [SMILES: Cc1cc(C)cc(-c2cc(-c3cnc(C(=O)NCC(=O)O)c(O)c3)on2)c1] |
| I-16 | 3-Hydroxy-5-(3- m-fluorophenylisoxazol- 5-yl)picolinoyl glycine | 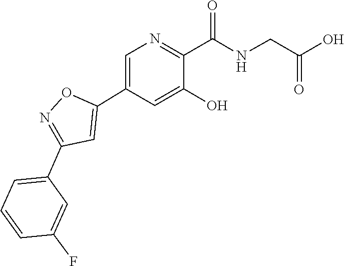 [SMILES: O=C(O)CNC(=O)c1ncc(-c2cc(-c3cccc(F)c3)no2)cc1O] |
| I-17 | 3-Hydroxy-5-(3- o-chlorophenylisoxazol- 5-yl)picolinoyl glycine | 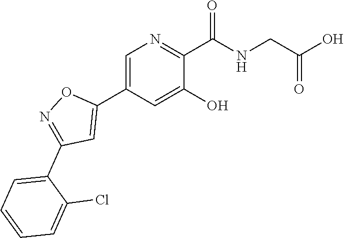 [SMILES: O=C(O)CNC(=O)c1ncc(-c2cc(-c3ccccc3Cl)no2)cc1O] |
| I-18 | 3-Hydroxy-5-(3- p-chlorophenylisoxazol- 5-yl)picolinoyl glycine | 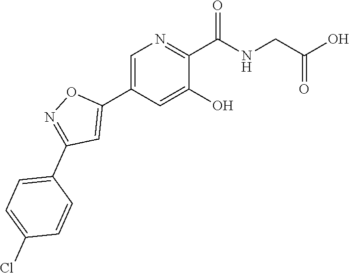 [SMILES: O=C(O)CNC(=O)c1ncc(-c2cc(-c3ccc(Cl)cc3)no2)cc1O] |
| I-19 | 3-Hydroxy-5-(3-m- dichlorophenylisoxazol- 5-yl)picolinoyl glycine | 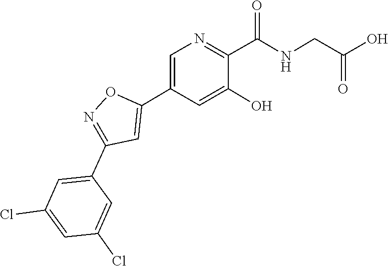 [SMILES: O=C(O)CNC(=O)c1ncc(-c2cc(-c3cc(Cl)cc(Cl)c3)no2)cc1O] |
| I-20 | 3-Hydroxy-5-(3- o-bromophenylisoxazol- 5-yl)picolinoyl glycine | 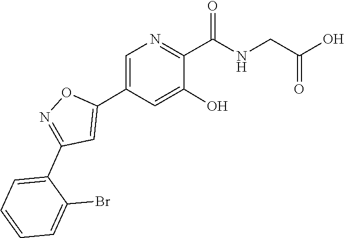 [SMILES: O=C(O)CNC(=O)c1ncc(-c2cc(-c3ccccc3Br)no2)cc1O] |
| I-21 | 3-Hydroxy-5-(3- p-bromophenylisoxazol- 5-yl)picolinoyl glycine | 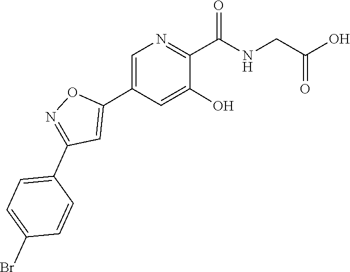 [SMILES: O=C(O)CNC(=O)c1ncc(-c2cc(-c3ccc(Br)cc3)no2)cc1O] |
| I-22 | 3-Hydroxy-5-(3-o- trifluoromethylphenylisoxazol- 5-yl)picolinoyl glycine | 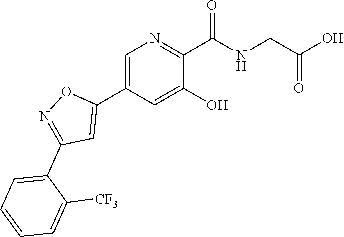 [SMILES: O=C(O)CNC(=O)c1ncc(-c2cc(-c3ccccc3C(F)(F)F)no2)cc1O] |
| I-23 | 3-Hydroxy-5-(3-p- trifluoromethylphenylisoxazol- 5-yl)picolinoyl glycine | 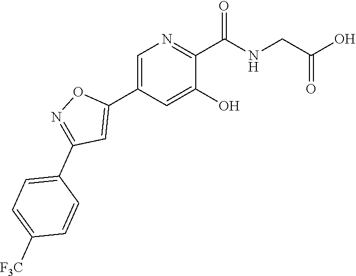 [SMILES: O=C(O)CNC(=O)c1ncc(-c2cc(-c3ccc(C(F)(F)F)cc3)no2)cc1O] |
| I-24 | 3-Hydroxy-5-(3-o- fluorophenylisoxazol- 5-yl)picolinoyl glycine | 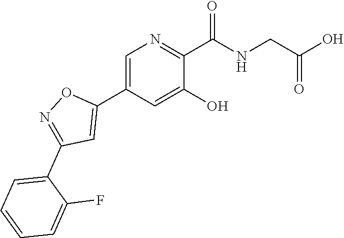 [SMILES: O=C(O)CNC(=O)c1ncc(-c2cc(-c3ccccc3F)no2)cc1O] |
| I-25 | 3-Hydroxy-5-(3- p-fluorophenylisoxazol- 5-yl)picolinoyl glycine | 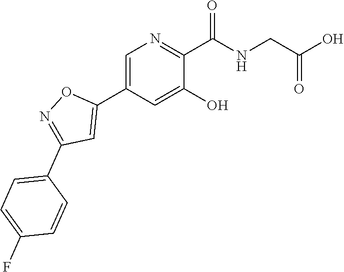 [SMILES: O=C(O)CNC(=O)c1ncc(-c2cc(-c3ccc(F)cc3)no2)cc1O] |
| I-26 | 3-Hydroxy-5-(3-m- difluorophenylisoxazol- 5-yl)picolinoyl glycine | 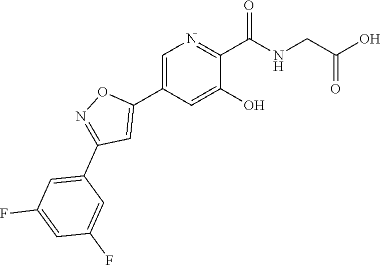 [SMILES: O=C(O)CNC(=O)c1ncc(-c2cc(-c3cc(F)cc(F)c3)no2)cc1O] |
| I-27 | 3-Hydroxy-5-(3-m-tert- butylphenylisoxazol 5-yl)picolinoyl glycine | 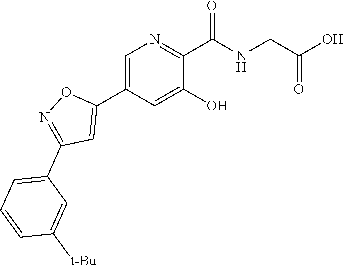 [SMILES: CC(C)(C)c1cccc(-c2cc(-c3cnc(C(=O)NCC(=O)O)c(O)c3)on2)c1] |
| I-28 | 3-Hydroxy-5-(3- phenylisoxazol-5-yl)-6- methylpicolinoyl glycine | 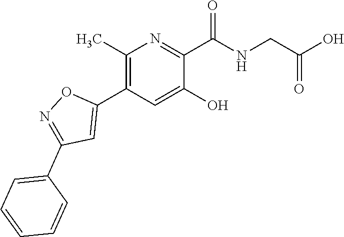 [SMILES: Cc1nc(C(=O)NCC(=O)O)c(O)cc1-c1cc(-c2ccccc2)no1] |
| I-29 | 3-Hydroxy-5-(4- methyl-3-phenylisoxazol- 5-yl)picolinoyl glycine | 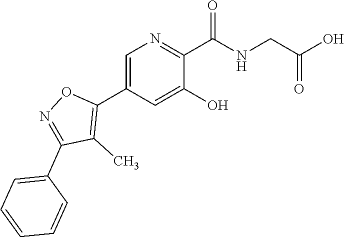 [SMILES: Cc1c(-c2ccccc2)noc1-c1cnc(C(=O)NCC(=O)O)c(O)c1] |
| I-30 | 3-Hydroxy-5-(4-tert-butyl-3- phenylisoxazol- 5-yl)picolinoyl glycine | 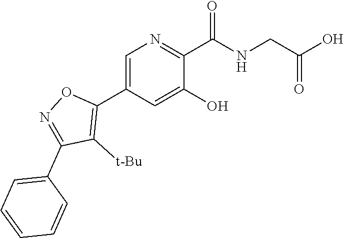 [SMILES: CC(C)(C)c1c(-c2ccccc2)noc1-c1cnc(C(=O)NCC(=O)O)c(O)c1] |
| I-31 | 3-Hydroxy-5-(4-cyclopropyl-3- phenylisoxazol- 5-yl)picolinoyl glycine | 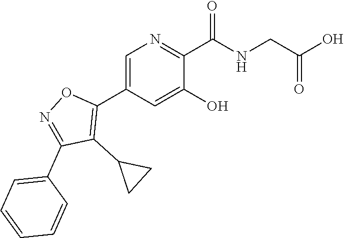 [SMILES: O=C(O)CNC(=O)c1ncc(-c2onc(-c3ccccc3)c2C2CC2)cc1O] |
| I-32 | 3-Hydroxy-5- (4-cyclohexyl-3- phenylisoxazol-5-yl) picolinoyl glycine | 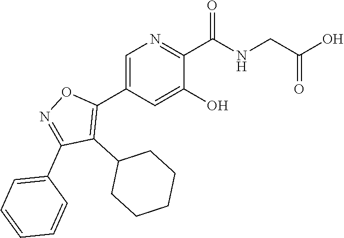 [SMILES: O=C(O)CNC(=O)c1ncc(-c2onc(-c3ccccc3)c2C2CCCCC2)cc1O] |
| I-33 | 3-Hydroxy-5- (3,4-diphenylisoxazol- 5-yl)picolinoyl glycine | 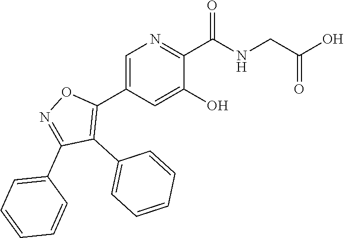 [SMILES: O=C(O)CNC(=O)c1ncc(-c2onc(-c3ccccc3)c2-c2ccccc2)cc1O] |

The pharmaceutically acceptable salt of the 3-hydroxy-5-(isoxazol-5-yl)picolinoyl glycine compound described above is a salt formed by the compound and an acid or a base; the acid is hydrochloric acid, hydrobromic acid, carbonic acid, sulfuric acid, phosphoric acid, methanesulfonic acid, benzenesulfonic acid, p-toluenesulfonic acid, naphthalenesulfonic acid, citric acid, malic acid, tartaric acid, lactic acid, pyruvic acid, acetic acid, maleic acid, succinic acid, fumaric acid, salicylic acid, phenylacetic acid, or mandelic acid; the base is an inorganic base comprising an alkali metal cation, an alkaline earth metal cation, or an ammonium cation salt, or an organic amine.

As a second aspect to which the present invention relates, the preparation method for the 3-hydroxy-5-(isoxazol-5-yl)picolinoyl glycine compound described above is as follows:
compound (III) is subjected to a cyclization reaction and a hydrolysis reaction to give compound (I);

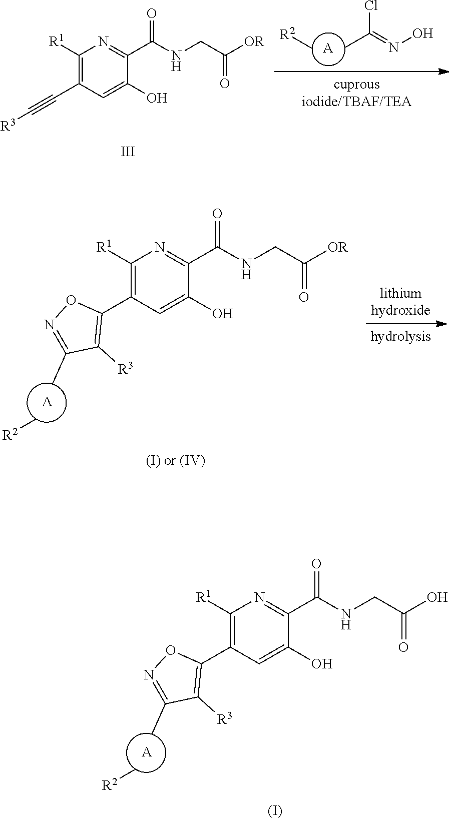

wherein A, R¹, R², and R³ are as previously defined; R represents hydrogen, C₁-C₄ aliphatic hydrocarbyl or benzyl;
a corresponding acid or base is subjected to a salt-forming reaction with compound (I) prepared by the above method to give the pharmaceutically acceptable salt of the compound.

As a third aspect to which the present invention relates, the pharmaceutical composition of the 3-hydroxy-5-(isoxazol-5-yl)picolinoyl glycine compound described above comprises the compound and a pharmaceutically acceptable carrier.

Pharmaceutically acceptable carriers can be added to the 3-hydroxy-5-(isoxazol-5-yl)picolinoyl glycine compound described above to prepare common pharmaceutical formulations, such as tablets, capsules, syrups, suspending agents, or injections, and common pharmaceutical adjuvants, such as perfuming agents, sweeteners, liquid/solid fillers, diluents, etc., can be added to the formulations.

As a fourth aspect to which the present invention relates, the 3-hydroxy-5-(isoxazol-5-yl)picolinoyl glycine compound or the pharmaceutical composition thereof described above is used as an inhibitor drug for a factor inhibiting HIF for treating a lipid metabolic disease, specifically, for treating obesity, hyperlipidemia, hypercholesterolemia, alcoholic fatty liver, nonalcoholic steatohepatitis (NASH), etc.

Beneficial Effects: The present invention has the following remarkable advantages compared with the prior art:
(1) such compounds and the pharmaceutical composition thereof can effectively inhibit factors inhibiting HIF, and the IC₅₀ value can optimally reach a nanomolar concentration level;
(2) such compounds and the pharmaceutical composition thereof are widely used and can be prepared into drugs for treating lipid metabolic diseases; the drugs can exert drug effects at a molecular level, a cellular level, and an animal level, significantly reducing triglyceride levels and restoring the fat metabolism level to normal;
(3) the preparation method for the compounds is simple, convenient, and easy to operate.

### BRIEF DESCRIPTION OF DRAWINGS

FIG. 1A shows the effects of the compounds of the present invention on lowering blood lipids in mice;

FIG. 1B shows the effects of the compounds on lowering cholesterol in mice;

FIG. 2 shows the effects of the compounds on weight loss in mice;

FIG. 3A shows the effects of the compounds on improving serum triglyceride in mice;

FIG. 3B shows the effects of the compounds on improving liver triglyceride in mice.

### DESCRIPTION OF EMBODIMENTS

The technical scheme of the present invention will be further described below with reference to the examples.

### Example 1: Preparation of Compound I-1

N-((5-(Trimethylsilyl)ethynyl)-3-hydroxypicolinoyl)glycine (200 mg, 0.69 mmol) was dissolved in methanol (10 mL), and then triethylamine (0.2 mL), cuprous iodide (20 mg), TBAF (1 mL), and N-hydroxybenzimidoyl chloride (127 mg, 0.82 mmol) were added. The mixture was conventionally heated to 50° C. for 4 h and reacted completely. After the reaction was completed, the mixture was filtered under vacuum to remove cuprous iodide and distilled under reduced pressure. Diluted hydrochloric acid (3 mmol) was added under an ice bath until a white solid was precipitated, and the mixture was filtered under vacuum and dried to give a white product (176 mg, total yield: 75.8%). m.p. 239.4-241.3° C. ¹H NMR (400 MHz, DMSO-d₆) δ 12.57 (s, 1H), 9.51 (t, J=6.0 Hz, 1H), 8.75 (d, J=1.8 Hz, 1H), 8.02-7.87 (m, 4H), 7.58 (dd, J=4.7, 2.6 Hz, 3H), 4.01 (d, J=6.1 Hz, 2H); EI-MS m/z: 340[M]⁺.

### Example 2: Preparation of Compound I-2

Methyl N-((5-(trimethylsilyl)ethynyl)-3-hydroxypicolinoyl)glycinate (200 mg, 0.69 mmol) was dissolved in methanol (10 mL), and then triethylamine (0.2 mL), cuprous iodide (20 mg), TBAF (1 mL), and N-hydroxy-m-methyl-benzimidoyl chloride (138 mg, 0.82 mmol) were added. The mixture was conventionally heated to 50° C. for 4 h and reacted completely. After the reaction was completed, the mixture was filtered under vacuum to remove cuprous iodide and distilled under reduced pressure. The crude product was separated and purified by silica gel column chromatography (petroleum ether:ethyl acetate=2:1) to give a white solid, which was dissolved in tetrahydrofuran (10 mL). A 1 M lithium hydroxide solution (3 mL) was added. The mixture was heated to 30° C. and reacted for 2 h and reacted completely. After the reaction was completed, the mixture was distilled under reduced pressure to remove tetrahydrofuran in the reaction solution. Diluted hydrochloric acid (3 mmol) was added under an ice bath until a white solid was precipitated, and the mixture was filtered under vacuum and dried to give a white product (102 mg, total yield: 44.1%). m.p. 250.8-253.1° C. ¹H NMR (400 MHz, DMSO-d₆) δ 9.20 (s, 1H), 8.68 (s, 1H), 7.87 (d, J=4.8 Hz, 2H), 7.78-7.69 (m, 2H), 7.41 (dd, J=32.6, 7.7 Hz, 2H), 3.80 (d, J=4.9 Hz, 2H); EI-MS m/z: 354[M]⁺.

### Example 3: Preparation of Compound I-3

Methyl N-((5-(trimethylsilyl)ethynyl)-3-hydroxypicolinoyl)glycinate (200 mg, 0.69 mmol) was dissolved in methanol (10 mL), and then triethylamine (0.2 mL), cuprous iodide (20 mg), TBAF (1 mL), and N-hydroxy-4-dibenzimidoyl chloride (189 mg, 0.82 mmol) were added. The mixture was conventionally heated to 50° C. for 4 h and reacted completely. After the reaction was completed, the mixture was filtered under vacuum to remove cuprous iodide and distilled under reduced pressure. The crude product was separated and purified by silica gel column chromatography (petroleum ether:ethyl acetate=2:1) to give a white solid, which was dissolved in tetrahydrofuran (10 mL). A 1 M lithium hydroxide solution (3 mL) was added. The mixture was heated to 30° C. and reacted for 2 h and reacted completely. After the reaction was completed, the mixture was distilled under reduced pressure to remove tetrahydrofuran in the reaction solution. Diluted hydrochloric acid (3 mmol) was added under an ice bath until a white solid was precipitated, and the mixture was filtered under vacuum and dried to give a white product (136 mg, total yield: 50.0%). m.p. 153.4-155.7° C. δ 12.57 (s, 1H), 9.45 (t, J=6.1 Hz, 1H), 8.74 (s, 1H), 8.01 (d, J=8.0 Hz, 2H), 7.97 (s, 1H), 7.93 (s, 1H), 7.88 (d, J=8.0 Hz, 2H), 7.77 (d, J=7.7 Hz, 2H), 7.52 (t, J=7.6 Hz, 2H), 7.43 (d, J=7.3 Hz, 1H), 4.01 (d, J=5.9 Hz, 2H); EI-MS m/z: 416[M]⁺.

### Example 4: Preparation of Compound I-4

Methyl N-((5-(trimethylsilyl)ethynyl)-3-hydroxypicolinoyl)glycinate (200 mg, 0.69 mmol) was dissolved in methanol (10 mL), and then triethylamine (0.2 mL), cuprous iodide (20 mg), TBAF (1 mL), and N-hydroxy-m-methoxybenzimidoyl chloride (152 mg, 0.82 mmol) were added. The mixture was conventionally heated to 50° C. for 4 h and reacted completely. After the reaction was completed, the mixture was filtered under vacuum to remove cuprous iodide and distilled under reduced pressure. The crude product was separated and purified by silica gel column chromatography (petroleum ether:ethyl acetate=2:1) to give a white solid, which was dissolved in tetrahydrofuran (10 mL). A 1 M lithium hydroxide solution (3 mL) was added. The mixture was heated to 30° C. and reacted for 2 h and reacted completely. After the reaction was completed, the mixture was distilled under reduced pressure to remove tetrahydrofuran in the reaction solution. Diluted hydrochloric acid (3 mmol) was added under an ice bath until a white solid was precipitated, and the mixture was filtered under vacuum and dried to give a white product (159 mg, total yield: 65.7%). 254.1-255.5° C. ¹H NMR (400 MHz, DMSO-d₆) δ 9.21-9.08 (m, 1H), 8.67 (d, J=18.0 Hz, 1H), 7.87 (t, J=15.4 Hz, 2H), 7.55-7.41 (m, 3H), 7.12 (s, 1H), 3.84 (d, J=8.3 Hz, 5H); EI-MS m/z: 370[M]⁺.

### Example 5: Preparation of Compound I-5

Methyl N-((5-(trimethylsilyl)ethynyl)-3-hydroxypicolinoyl)glycinate (200 mg, 0.69 mmol) was dissolved in methanol (10 mL), and then triethylamine (0.2 mL), cuprous iodide (20 mg), TBAF (1 mL), and N-hydroxy-m-chlorobenzimidoyl chloride (154 mg, 0.82 mmol) were added. The mixture was conventionally heated to 50° C. for 4 h and reacted completely. After the reaction was completed, the mixture was filtered under vacuum to remove cuprous iodide and distilled under reduced pressure. The crude product was separated and purified by silica gel column chromatography (petroleum ether:ethyl acetate=2:1) to give a white solid, which was dissolved in tetrahydrofuran (10 mL). A 1 M lithium hydroxide solution (3 mL) was added. The mixture was heated to 30° C. and reacted for 2 h and reacted completely. After the reaction was completed, the mixture was distilled under reduced pressure to remove tetrahydrofuran in the reaction solution. Diluted hydrochloric acid (3 mmol) was added under an ice bath until a white solid was precipitated, and the mixture was filtered under vacuum and dried to give a white product (112 mg, total yield: 45.8%). m.p. 210.7-212.6° C. ¹H NMR (400 MHz, DMSO-d₆) δ 9.41 (t, J=6.0 Hz, 1H), 8.70 (s, 1H), 8.03-7.86 (m, 4H), 7.62 (d, J=6.8 Hz, 2H), 3.97 (d, J=5.8 Hz, 2H). ¹H NMR (400 MHz, DMSO-d₆) δ 9.34 (s, 1H), 8.70 (s, 1H), 7.97 (s, 2H), 7.92-7.87 (m, 2H), 7.62 (d, J=6.8 Hz, 2H), 3.91 (d, J=5.5 Hz, 2H); EI-MS m/z: 374[M]⁺.

### Example 6: Preparation of Compound I-6

Methyl N-((5-(trimethylsilyl)ethynyl)-3-hydroxypicolinoyl)glycinate (200 mg, 0.69 mmol) was dissolved in methanol (10 mL), and then triethylamine (0.2 mL), cuprous iodide (20 mg), TBAF (1 mL), and N-hydroxy-m-bromobenzimidoyl chloride (190 mg, 0.82 mmol) were added. The mixture was conventionally heated to 50° C. for 4 h and reacted completely. After the reaction was completed, the mixture was filtered under vacuum to remove cuprous iodide and distilled under reduced pressure. The crude product was separated and purified by silica gel column chromatography (petroleum ether:ethyl acetate=2:1) to give a white solid, which was dissolved in tetrahydrofuran (10 mL). A 1 M lithium hydroxide solution (3 mL) was added. The mixture was heated to 30° C. and reacted for 2 h and reacted completely. After the reaction was completed, the mixture was distilled under reduced pressure to remove tetrahydrofuran in the reaction solution. Diluted hydrochloric acid (3 mmol) was added under an ice bath until a white solid was precipitated, and the mixture was filtered under vacuum and dried to give a white product (133 mg, total yield: 48.8%). m.p. 213.0-215.3° C. ¹H NMR (400 MHz, DMSO-d₆) δ 12.57 (s, 1H), 9.48 (t, J=6.1 Hz, 1H), 8.71 (d, J=1.8 Hz, 1H), 8.10 (d, J=2.0 Hz, 1H), 7.99 (s, 1H), 7.94 (d, J=7.8 Hz, 1H), 7.90 (d, J=1.8 Hz, 1H), 7.77 (dd, J=8.0, 2.0 Hz, 1H), 7.55 (t, J=7.9 Hz, 1H), 4.01 (d, J=6.1 Hz, 2H); EI-MS m/z: 417[M]⁺.

### Example 7: Preparation of Compound I-7

Methyl N-((5-(trimethylsilyl)ethynyl)-3-hydroxypicolinoyl)glycinate (200 mg, 0.69 mmol) was dissolved in methanol (10 mL), and then triethylamine (0.2 mL), cuprous iodide (20 mg), TBAF (1 mL), and N-hydroxycyclohexanecarbimidoyl chloride (132 mg, 0.82 mmol) were added. The mixture was conventionally heated to 50° C. for 4 h and reacted completely. After the reaction was completed, the mixture was filtered under vacuum to remove cuprous iodide and distilled under reduced pressure. The crude product was separated and purified by silica gel column chromatography (petroleum ether:ethyl acetate=2:1) to give a white solid, which was dissolved in tetrahydrofuran (10 mL). A 1 M lithium hydroxide solution (3 mL) was added. The mixture was heated to 30° C. and reacted for 2 h and reacted completely. After the reaction was completed, the mixture was distilled under reduced pressure to remove tetrahydrofuran in the reaction solution. Diluted hydrochloric acid (3 mmol) was added under an ice bath until a white solid was precipitated, and the mixture was filtered under vacuum and dried to give a white product (176 mg, total yield: 77.8%). m.p. 247.2-249.4° C. ¹H NMR (400 MHz, DMSO-d₆) δ 12.53 (s, 1H), 9.44 (t, J=6.1 Hz, 1H), 8.65 (d, J=1.8 Hz, 1H), 7.84 (d, J=1.9 Hz, 1H), 7.33 (s, 1H), 4.00 (d, J=6.1 Hz, 2H), 2.80 (tt, J=11.3, 3.7 Hz, 1H), 1.95 (dt, J=13.5, 3.2 Hz, 2H), 1.78 (dt, J=12.4, 3.3 Hz, 2H), 1.54-1.19 (m, 6H); EI-MS m/z: 346[M]⁺.

### Example 8: Preparation of Compound I-8

Methyl N-((5-(trimethylsilyl)ethynyl)-3-hydroxypicolinoyl)glycinate (200 mg, 0.69 mmol) was dissolved in methanol (10 mL), and then triethylamine (0.2 mL), cuprous iodide (20 mg), TBAF (1 mL), and N-hydroxycyclopropanecarbimidoyl chloride (98 mg, 0.82 mmol) were added. The mixture was conventionally heated to 50° C. for 4 h and reacted completely. After the reaction was completed, the mixture was filtered under vacuum to remove cuprous iodide and distilled under reduced pressure. The crude product was separated and purified by silica gel column chromatography (petroleum ether:ethyl acetate=2:1) to give a white solid, which was dissolved in tetrahydrofuran (10 mL). A 1 M lithium hydroxide solution (3 mL) was added. The mixture was heated to 30° C. and reacted for 2 h and reacted completely. After the reaction was completed, the mixture was distilled under reduced pressure to remove tetrahydrofuran in the reaction solution. Diluted hydrochloric acid (3 mmol) was added under an ice bath until a white solid was precipitated, and the mixture was filtered under vacuum and dried to give a white product (132 mg, total yield: 66.4%). m.p. 232.7-235.3° C. ¹H NMR (400 MHz, DMSO-d₆) δ 12.54 (s, 1H), 9.42 (t, J=6.1 Hz, 1H), 8.61 (d, J=1.9 Hz, 1H), 7.80 (d, J=1.8 Hz, 1H), 7.11 (s, 1H), 3.99 (d, J=6.0 Hz, 2H), 2.10 (tt, J=8.6, 4.9 Hz, 1H), 1.15-1.03 (m, 2H), 0.84 (dt, J=6.8, 4.4 Hz, 2H); EI-MS m/z: 304[M]⁺.

### Example 9: Preparation of Compound I-9

Methyl N-((5-(trimethylsilyl)ethynyl)-3-hydroxypicolinoyl)glycinate (200 mg, 0.69 mmol) was dissolved in methanol (10 mL), and then triethylamine (0.2 mL), cuprous iodide (20 mg), TBAF (1 mL), and N-hydroxy-m-cyanobenzimidoyl chloride (148 mg, 0.82 mmol) were added. The mixture was conventionally heated to 50° C. for 4 h and reacted completely. After the reaction was completed, the mixture was filtered under vacuum to remove cuprous iodide and distilled under reduced pressure. The crude product was separated and purified by silica gel column chromatography (petroleum ether:ethyl acetate=2:1) to give a white solid, which was dissolved in tetrahydrofuran (10 mL). A 1 M lithium hydroxide solution (3 mL) was added. The mixture was heated to 30° C. and reacted for 2 h and reacted completely. After the reaction was completed, the mixture was distilled under reduced pressure to remove tetrahydrofuran in the reaction solution. Diluted hydrochloric acid (3 mmol) was added under an ice bath until a white solid was precipitated, and the mixture was filtered under vacuum and dried to give a white product (154 mg, total yield: 64.6%). m.p. 205.4-208.1° C. ¹H NMR (400 MHz, DMSO-d₆) δ 12.62 (s, 1H), 9.51 (t, J=6.1 Hz, 1H), 8.72 (d, J=1.8 Hz, 1H), 8.38 (d, J=1.8 Hz, 1H), 8.27 (dt, J=8.0, 1.5 Hz, 1H), 8.05 (d, J=7.2 Hz, 2H), 7.91 (d, J=1.8 Hz, 1H), 7.81 (t, J=7.8 Hz, 1H), 4.01 (d, J=6.1 Hz, 2H); EI-MS m/z: 365[M]⁺.

### Example 10: Preparation of Compound I-10

Methyl N-((5-(trimethylsilyl)ethynyl)-3-hydroxypicolinoyl)glycinate (200 mg, 0.69 mmol) was dissolved in methanol (10 mL), and then triethylamine (0.2 mL), cuprous iodide (20 mg), TBAF (1 mL), and N-hydroxy-m-trifluoromethylbenzimidoyl chloride (183 mg, 0.82 mmol) were added. The mixture was conventionally heated to 50° C. for 4 h and reacted completely. After the reaction was completed, the mixture was filtered under vacuum to remove cuprous iodide and distilled under reduced pressure. The crude product was separated and purified by silica gel column chromatography (petroleum ether:ethyl acetate=2:1) to give a white solid, which was dissolved in tetrahydrofuran (10 mL). A 1 M lithium hydroxide solution (3 mL) was added. The mixture was heated to 30° C. and reacted for 2 h and reacted completely. After the reaction was completed, the mixture was distilled under reduced pressure to remove tetrahydrofuran in the reaction solution. Diluted hydrochloric acid (3 mmol) was added under an ice bath until a white solid was precipitated, and the mixture was filtered under vacuum and dried to give a white product (141 mg, total yield: 52.9%). m.p. 245.2-247.3° C. ¹H NMR (400 MHz, DMSO-d₆) δ 12.57 (s, 1H), 9.50 (t, J=6.1 Hz, 1H), 8.74 (d, J=1.8 Hz, 1H), 8.28-8.21 (m, 2H), 8.10 (s, 1H), 7.98-7.92 (m, 2H), 7.84 (t, J=7.8 Hz, 1H), 4.02 (d, J=6.1 Hz, 2H); EI-MS m/z: 408[M]⁺.

### Example 11: Preparation of Compound I-11

Methyl N-((5-(trimethylsilyl)ethynyl)-3-hydroxypicolinoyl)glycinate (200 mg, 0.69 mmol) was dissolved in methanol (10 mL), and then triethylamine (0.2 mL), cuprous iodide (20 mg), TBAF (1 mL), and N-hydroxy-1-naphthylcarbimidoyl chloride (168 mg, 0.82 mmol) were added. The mixture was conventionally heated to 50° C. for 4 h and reacted completely. After the reaction was completed, the mixture was filtered under vacuum to remove cuprous iodide and distilled under reduced pressure. The crude product was separated and purified by silica gel column chromatography (petroleum ether:ethyl acetate=2:1) to give a white solid, which was dissolved in tetrahydrofuran (10 mL). A 1 M lithium hydroxide solution (3 mL) was added. The mixture was heated to 30° C. and reacted for 2 h and reacted completely. After the reaction was completed, the mixture was distilled under reduced pressure to remove tetrahydrofuran in the reaction solution. Diluted hydrochloric acid (3 mmol) was added under an ice bath until a white solid was precipitated, and the mixture was filtered under vacuum and dried to give a white product (109 mg, total yield: 44.8%). m.p. 180.5-183.4° C. ¹H NMR (400 MHz, DMSO-d₆) δ 12.55 (s, 1H), 9.51 (t, J=6.1 Hz, 1H), 8.81 (d, J=1.8 Hz, 1H), 8.51-8.43 (m, 1H), 8.15 (d, J=8.2 Hz, 1H), 8.11-8.07 (m, 1H), 8.02 (d, J=1.8 Hz, 1H), 7.92-7.86 (m, 2H), 7.74-7.62 (m, 3H), 4.03 (d, J=6.1 Hz, 2H), 2.51 (d, J=5.7 Hz, 4H); EI-MS m/z: 390[M]⁺.

### Example 12: Preparation of Compound I-12

Methyl N-((5-(trimethylsilyl)ethynyl)-3-hydroxypicolinoyl)glycinate (200 mg, 0.69 mmol) was dissolved in methanol (10 mL), and then triethylamine (0.2 mL), cuprous iodide (20 mg), TBAF (1 mL), and N-hydroxy-2-naphthylcarbimidoyl chloride (168 mg, 0.82 mmol) were added. The mixture was conventionally heated to 50° C. for 4 h and reacted completely. After the reaction was completed, the mixture was filtered under vacuum to remove cuprous iodide and distilled under reduced pressure. The crude product was separated and purified by silica gel column chromatography (petroleum ether:ethyl acetate=2:1) to give a white solid, which was dissolved in tetrahydrofuran (10 mL). A 1 M lithium hydroxide solution (3 mL) was added. The mixture was heated to 30° C. and reacted for 2 h and reacted completely. After the reaction was completed, the mixture was distilled under reduced pressure to remove tetrahydrofuran in the reaction solution. Diluted hydrochloric acid (3 mmol) was added under an ice bath until a white solid was precipitated, and the mixture was filtered under vacuum and dried to give a white product (125 mg, total yield: 49.0%). m.p. 259.3-261.4° C. ¹H NMR (400 MHz, DMSO-d₆) δ 9.26 (s, 1H), 8.71 (s, 1H), 8.49 (s, 1H), 8.10-8.00 (m, 5H), 7.89 (s, 1H), 7.63 (dt, J=6.8, 3.4 Hz, 2H), 3.86 (d, J=5.2 Hz, 2H); EI-MS m/z: 390[M]⁺.

### Example 13: Preparation of Compound I-13

Methyl N-((5-(trimethylsilyl)ethynyl)-3-hydroxypicolinoyl)glycinate (200 mg, 0.69 mmol) was dissolved in methanol (10 mL), and then triethylamine (0.2 mL), cuprous iodide (20 mg), TBAF (1 mL), and N-hydroxy-o-methylbenzimidoyl chloride (138 mg, 0.82 mmol) were added. The mixture was conventionally heated to 50° C. for 4 h and reacted completely. After the reaction was completed, the mixture was filtered under vacuum to remove cuprous iodide and distilled under reduced pressure. The crude product was separated and purified by silica gel column chromatography (petroleum ether:ethyl acetate=2:1) to give a white solid, which was dissolved in tetrahydrofuran (10 mL). A 1 M lithium hydroxide solution (3 mL) was added. The mixture was heated to 30° C. and reacted for 2 h and reacted completely. After the reaction was completed, the mixture was distilled under reduced pressure to remove tetrahydrofuran in the reaction solution. Diluted hydrochloric acid (3 mmol) was added under an ice bath until a white solid was precipitated, and the mixture was filtered under vacuum and dried to give a white product (131 mg, total yield: 56.6%). m.p. 212.2-214.9° C. ¹H NMR (400 MHz, DMSO-d₆) δ 9.45-9.36 (m, 1H), 8.75 (d, J=1.8 Hz, 1H), 7.96 (d, J=1.8 Hz, 1H), 7.75 (s, 1H), 7.63 (d, J=7.5 Hz, 1H), 7.47-7.35 (m, 3H), 3.95 (d, J=5.9 Hz, 2H), 2.52 (s, 3H); EI-MS m/z: 354[M]⁺.

### Example 14: Preparation of Compound I-14

Methyl N-((5-(trimethylsilyl)ethynyl)-3-hydroxypicolinoyl)glycinate (200 mg, 0.69 mmol) was dissolved in methanol (10 mL), and then triethylamine (0.2 mL), cuprous iodide (20 mg), TBAF (1 mL), and N-hydroxy-p-methylbenzimidoyl chloride (138 mg, 0.82 mmol) were added. The mixture was conventionally heated to 50° C. for 4 h and reacted completely. After the reaction was completed, the mixture was filtered under vacuum to remove cuprous iodide and distilled under reduced pressure. The crude product was separated and purified by silica gel column chromatography (petroleum ether:ethyl acetate=2:1) to give a white solid, which was dissolved in tetrahydrofuran (10 mL). A 1 M lithium hydroxide solution (3 mL) was added. The mixture was heated to 30° C. and reacted for 2 h and reacted completely. After the reaction was completed, the mixture was distilled under reduced pressure to remove tetrahydrofuran in the reaction solution. Diluted hydrochloric acid (3 mmol) was added under an ice bath until a white solid was precipitated, and the mixture was filtered under vacuum and dried to give a white product (157 mg, total yield: 67.8%). m.p. 260.8-262.7° C. ¹H NMR (400 MHz, DMSO-d₆) δ 9.48-9.35 (m, 1H), 8.72 (s, 1H), 7.96-7.86 (m, 2H), 7.82 (d, J=7.8 Hz, 2H), 7.39 (d, J=7.8 Hz, 2H), 3.95 (d, J=5.8 Hz, 2H), 2.39 (s, 3H); EI-MS m/z: 354[M]⁺.

### Example 15: Preparation of Compound I-15

Methyl N-((5-(trimethylsilyl)ethynyl)-3-hydroxypicolinoyl)glycinate (200 mg, 0.69 mmol) was dissolved in methanol (10 mL), and then triethylamine (0.2 mL), cuprous iodide (20 mg), TBAF (1 mL), and N-hydroxy-m-dimethylbenzimidoyl chloride (150 mg, 0.82 mmol) were added. The mixture was conventionally heated to 50° C. for 4 h and reacted completely. After the reaction was completed, the mixture was filtered under vacuum to remove cuprous iodide and distilled under reduced pressure. The crude product was separated and purified by silica gel column chromatography (petroleum ether:ethyl acetate=2:1) to give a white solid, which was dissolved in tetrahydrofuran (10 mL). A 1 M lithium hydroxide solution (3 mL) was added. The mixture was heated to 30° C. and reacted for 2 h and reacted completely. After the reaction was completed, the mixture was distilled under reduced pressure to remove tetrahydrofuran in the reaction solution. Diluted hydrochloric acid (3 mmol) was added under an ice bath until a white solid was precipitated, and the mixture was filtered under vacuum and dried to give a white product (140 mg, total yield: 58.2%). m.p. 277.6-279.8° C. ¹H NMR (400 MHz, DMSO-d₆) δ 12.56 (s, 1H), 9.50 (t, J=6.1 Hz, 1H), 8.73 (d, J=1.9 Hz, 1H), 7.92 (d, J=2.0 Hz, 2H), 7.55 (s, 2H), 7.19 (s, 1H), 4.01 (d, J=6.1 Hz, 2H), 2.37 (s, 6H); EI-MS m/z: 368[M]⁺.

### Example 16: Preparation of Compound I-16

Methyl N-((5-(trimethylsilyl)ethynyl)-3-hydroxypicolinoyl)glycinate (200 mg, 0.69 mmol) was dissolved in methanol (10 mL), and then triethylamine (0.2 mL), cuprous iodide (20 mg), TBAF (1 mL), and N-hydroxy-m-fluorobenzimidoyl chloride (142 mg, 0.82 mmol) were added. The mixture was conventionally heated to 50° C. for 4 h and reacted completely. After the reaction was completed, the mixture was filtered under vacuum to remove cuprous iodide and distilled under reduced pressure. The crude product was separated and purified by silica gel column chromatography (petroleum ether:ethyl acetate=2:1) to give a white solid, which was dissolved in tetrahydrofuran (10 mL). A 1 M lithium hydroxide solution (3 mL) was added. The mixture was heated to 30° C. and reacted for 2 h and reacted completely. After the reaction was completed, the mixture was distilled under reduced pressure to remove tetrahydrofuran in the reaction solution. Diluted hydrochloric acid (3 mmol) was added under an ice bath until a white solid was precipitated, and the mixture was filtered under vacuum and dried to give a white product (166 mg, total yield: 70.9%). m.p. 236.7-238.9° C. ¹H NMR (400 MHz, DMSO-d₆) δ 12.62 (s, 1H), 9.47 (t, J=6.1 Hz, 1H), 8.71 (d, J=1.8 Hz, 1H), 7.97 (s, 1H), 7.91 (d, J=1.8 Hz, 1H), 7.80-7.72 (m, 2H), 7.64 (td, J=8.0, 5.9 Hz, 1H), 7.42 (td, J=8.6, 2.7 Hz, 1H), 4.00 (d, J=6.0 Hz, 2H); EI-MS m/z: 358[M]⁺.

### Example 17: Preparation of Compound I-17

Methyl N-((5-(trimethylsilyl)ethynyl)-3-hydroxypicolinoyl)glycinate (200 mg, 0.69 mmol) was dissolved in methanol (10 mL), and then triethylamine (0.2 mL), cuprous iodide (20 mg), TBAF (1 mL), and N-hydroxy-o-chlorobenzimidoyl chloride (155 mg, 0.82 mmol) were added. The mixture was conventionally heated to 50° C. for 4 h and reacted completely. After the reaction was completed, the mixture was filtered under vacuum to remove cuprous iodide and distilled under reduced pressure. The crude product was separated and purified by silica gel column chromatography (petroleum ether:ethyl acetate=2:1) to give a white solid, which was dissolved in tetrahydrofuran (10 mL). A 1 M lithium hydroxide solution (3 mL) was added. The mixture was heated to 30° C. and reacted for 2 h and reacted completely. After the reaction was completed, the mixture was distilled under reduced pressure to remove tetrahydrofuran in the reaction solution. Diluted hydrochloric acid (3 mmol) was added under an ice bath until a white solid was precipitated, and the mixture was filtered under vacuum and dried to give a white product (116 mg, total yield: 47.4%). m.p. 255.9-258.0° C. ¹H NMR (400 MHz, DMSO-d₆) δ 9.44 (t, J=6.0 Hz, 1H), 8.77 (d, J=1.9 Hz, 1H), 8.01 (d, J=1.8 Hz, 1H), 7.81 (s, 1H), 7.77 (dd, J=7.6, 1.9 Hz, 1H), 7.71 (d, J=7.8 Hz, 1H), 7.62-7.53 (m, 2H), 3.97 (d, J=5.9 Hz, 2H); EI-MS m/z: 374[M]⁺.

### Example 18: Preparation of Compound I-18

Methyl N-((5-(trimethylsilyl)ethynyl)-3-hydroxypicolinoyl)glycinate (200 mg, 0.69 mmol) was dissolved in methanol (10 mL), and then triethylamine (0.2 mL), cuprous iodide (20 mg), TBAF (1 mL), and N-hydroxy-p-chlorobenzimidoyl chloride (155 mg, 0.82 mmol) were added. The mixture was conventionally heated to 50° C. for 4 h and reacted completely. After the reaction was completed, the mixture was filtered under vacuum to remove cuprous iodide and distilled under reduced pressure. The crude product was separated and purified by silica gel column chromatography (petroleum ether:ethyl acetate=2:1) to give a white solid, which was dissolved in tetrahydrofuran (10 mL). A 1 M lithium hydroxide solution (3 mL) was added. The mixture was heated to 30° C. and reacted for 2 h and reacted completely. After the reaction was completed, the mixture was distilled under reduced pressure to remove tetrahydrofuran in the reaction solution. Diluted hydrochloric acid (3 mmol) was added under an ice bath until a white solid was precipitated, and the mixture was filtered under vacuum and dried to give a white product (169 mg, total yield: 69.1%). m.p. 247.6-249.5° C. ¹H NMR (400 MHz, DMSO-d₆) δ 9.17 (s, 1H), 8.68 (s, 1H), 7.94 (t, J=7.6 Hz, 3H), 7.87 (s, 1H), 7.66 (d, J=8.1 Hz, 2H), 3.74 (s, 2H); EI-MS m/z: 374[M]⁺.

### Example 19: Preparation of Compound I-19

Methyl N-((5-(trimethylsilyl)ethynyl)-3-hydroxypicolinoyl)glycinate (200 mg, 0.69 mmol) was dissolved in methanol (10 mL), and then triethylamine (0.2 mL), cuprous iodide (20 mg), TBAF (1 mL), and N-hydroxy-m-dichlorobenzimidoyl chloride (183 mg, 0.82 mmol) were added. The mixture was conventionally heated to 50° C. for 4 h and reacted completely. After the reaction was completed, the mixture was filtered under vacuum to remove cuprous iodide and distilled under reduced pressure. The crude product was separated and purified by silica gel column chromatography (petroleum ether:ethyl acetate=2:1) to give a white solid, which was dissolved in tetrahydrofuran (10 mL). A 1 M lithium hydroxide solution (3 mL) was added. The mixture was heated to 30° C. and reacted for 2 h and reacted completely. After the reaction was completed, the mixture was distilled under reduced pressure to remove tetrahydrofuran in the reaction solution. Diluted hydrochloric acid (3 mmol) was added under an ice bath until a white solid was precipitated, and the mixture was filtered under vacuum and dried to give a white product (143 mg, total yield: 53.6%). m.p. 222.8-224.9° C. ¹H NMR (400 MHz, DMSO-d₆) δ 9.31 (s, 1H), 8.64 (s, 1H), 8.01 (s, 1H), 7.93 (d, J=2.0 Hz, 2H), 7.82 (d, J=2.2 Hz, 2H), 3.89 (d, J=5.4 Hz, 2H); EI-MS m/z: 408[M]⁺.

### Example 20: Preparation of Compound I-20

Methyl N-((5-(trimethylsilyl)ethynyl)-3-hydroxypicolinoyl)glycinate (200 mg, 0.69 mmol) was dissolved in methanol (10 mL), and then triethylamine (0.2 mL), cuprous iodide (20 mg), TBAF (1 mL), and N-hydroxy-o-bromobenzimidoyl chloride (191 mg, 0.82 mmol) were added. The mixture was conventionally heated to 50° C. for 4 h and reacted completely. After the reaction was completed, the mixture was filtered under vacuum to remove cuprous iodide and distilled under reduced pressure. The crude product was separated and purified by silica gel column chromatography (petroleum ether:ethyl acetate=2:1) to give a white solid, which was dissolved in tetrahydrofuran (10 mL). A 1 M lithium hydroxide solution (3 mL) was added. The mixture was heated to 30° C. and reacted for 2 h and reacted completely. After the reaction was completed, the mixture was distilled under reduced pressure to remove tetrahydrofuran in the reaction solution. Diluted hydrochloric acid (3 mmol) was added under an ice bath until a white solid was precipitated, and the mixture was filtered under vacuum and dried to give a white product (124 mg, total yield: 45.4%). m.p. 277.7-279.5° C. ¹H NMR (400 MHz, DMSO-d₆) δ 9.27-9.12 (m, 1H), 8.72 (s, 1H), 7.94 (s, 1H), 7.86 (d, J=7.9 Hz, 1H), 7.77-7.65 (m, 2H), 7.54 (dt, J=27.3, 7.6 Hz, 2H), 3.79 (d, J=5.1 Hz, 2H); EI-MS m/z: 418[M]⁺.

### Example 21: Preparation of Compound I-21

Methyl N-((5-(trimethylsilyl)ethynyl)-3-hydroxypicolinoyl)glycinate (200 mg, 0.69 mmol) was dissolved in methanol (10 mL), and then triethylamine (0.2 mL), cuprous iodide (20 mg), TBAF (1 mL), and N-hydroxy-p-bromobenzimidoyl chloride (191 mg, 0.82 mmol) were added. The mixture was conventionally heated to 50° C. for 4 h and reacted completely. After the reaction was completed, the mixture was filtered under vacuum to remove cuprous iodide and distilled under reduced pressure. The crude product was separated and purified by silica gel column chromatography (petroleum ether:ethyl acetate=2:1) to give a white solid, which was dissolved in tetrahydrofuran (10 mL). A 1 M lithium hydroxide solution (3 mL) was added. The mixture was heated to 30° C. and reacted for 2 h and reacted completely. After the reaction was completed, the mixture was distilled under reduced pressure to remove tetrahydrofuran in the reaction solution. Diluted hydrochloric acid (3 mmol) was added under an ice bath until a white solid was precipitated, and the mixture was filtered under vacuum and dried to give a white product (149 mg, total yield: 55.9%). m.p. 215.0-216.3° C. ¹H NMR (400 MHz, DMSO-d₆) δ 9.38-9.23 (m, 1H), 8.69 (s, 1H), 7.89 (t, J=12.5 Hz, 4H), 7.80 (d, J=7.9 Hz, 2H), 3.92-3.84 (m, 2H); EI-MS m/z: 408[M]⁺.

### Example 22: Preparation of Compound I-22

Methyl N-((5-(trimethylsilyl)ethynyl)-3-hydroxypicolinoyl)glycinate (200 mg, 0.69 mmol) was dissolved in methanol (10 mL), and then triethylamine (0.2 mL), cuprous iodide (20 mg), TBAF (1 mL), and N-hydroxy-o-trifluoromethylbenzimidoyl chloride (183 mg, 0.82 mmol) were added. The mixture was conventionally heated to 50° C. for 4 h and reacted completely. After the reaction was completed, the mixture was filtered under vacuum to remove cuprous iodide and distilled under reduced pressure. The crude product was separated and purified by silica gel column chromatography (petroleum ether:ethyl acetate=2:1) to give a white solid, which was dissolved in tetrahydrofuran (10 mL). A 1 M lithium hydroxide solution (3 mL) was added. The mixture was heated to 30° C. and reacted for 2 h and reacted completely. After the reaction was completed, the mixture was distilled under reduced pressure to remove tetrahydrofuran in the reaction solution. Diluted hydrochloric acid (3 mmol) was added under an ice bath until a white solid was precipitated, and the mixture was filtered under vacuum and dried to give a white product (171 mg, total yield: 64.1%). m.p. 278.9-281.1° C. ¹H NMR (400 MHz, DMSO-d₆) δ 12.57 (s, 1H), 9.57-9.45 (m, 1H), 8.84-8.73 (m, 1H), 8.06-7.98 (m, 2H), 7.84 (dd, J=18.0, 7.4 Hz, 2H), 7.76 (d, J=7.9 Hz, 1H), 7.64 (d, J=3.0 Hz, 1H), 4.02 (d, J=5.6 Hz, 2H); EI-MS m/z: 408 [M]⁺.

### Example 23: Preparation of Compound I-23

Methyl N-((5-(trimethylsilyl)ethynyl)-3-hydroxypicolinoyl)glycinate (200 mg, 0.69 mmol) was dissolved in methanol (10 mL), and then triethylamine (0.2 mL), cuprous iodide (20 mg), TBAF (1 mL), and N-hydroxy-p-trifluoromethylbenzimidoyl chloride (183 mg, 0.82 mmol) were added. The mixture was conventionally heated to 50° C. for 4 h and reacted completely. After the reaction was completed, the mixture was filtered under vacuum to remove cuprous iodide and distilled under reduced pressure. The crude product was separated and purified by silica gel column chromatography (petroleum ether:ethyl acetate=2:1) to give a white solid, which was dissolved in tetrahydrofuran (10 mL). A 1 M lithium hydroxide solution (3 mL) was added. The mixture was heated to 30° C. and reacted for 2 h and reacted completely. After the reaction was completed, the mixture was distilled under reduced pressure to remove tetrahydrofuran in the reaction solution. Diluted hydrochloric acid (3 mmol) was added under an ice bath until a white solid was precipitated, and the mixture was filtered under vacuum and dried to give a white product (120 mg, total yield: 45.0%). m.p. 288.8-291.2° C. ¹H NMR (400 MHz, DMSO-d₆) δ 9.25-9.00 (m, 1H), 8.61 (s, 1H), 8.13 (d, J=7.3 Hz, 2H), 7.94 (d, J=8.6 Hz, 3H), 7.80 (s, 1H), 3.72-3.64 (m, 2H); EI-MS m/z: 408[M]⁺.

### Example 24: Preparation of Compound I-24

Methyl N-((5-(trimethylsilyl)ethynyl)-3-hydroxypicolinoyl)glycinate (200 mg, 0.69 mmol) was dissolved in methanol (10 mL), and then triethylamine (0.2 mL), cuprous iodide (20 mg), TBAF (1 mL), and N-hydroxy-o-fluorobenzimidoyl chloride (142 mg, 0.82 mmol) were added. The mixture was conventionally heated to 50° C. for 4 h and reacted completely. After the reaction was completed, the mixture was filtered under vacuum to remove cuprous iodide and distilled under reduced pressure. The crude product was separated and purified by silica gel column chromatography (petroleum ether:ethyl acetate=2:1) to give a white solid, which was dissolved in tetrahydrofuran (10 mL). A 1 M lithium hydroxide solution (3 mL) was added. The mixture was heated to 30° C. and reacted for 2 h and reacted completely. After the reaction was completed, the mixture was distilled under reduced pressure to remove tetrahydrofuran in the reaction solution. Diluted hydrochloric acid (3 mmol) was added under an ice bath until a white solid was precipitated, and the mixture was filtered under vacuum and dried to give a white product (126 mg, total yield: 53.8%). m.p. 241.5-243.1° C. ¹H NMR (400 MHz, DMSO-d₆) δ 9.33 (s, 1H), 8.80-8.72 (m, 1H), 8.01-7.91 (m, 2H), 7.78 (s, 1H), 7.63 (d, J=2.8 Hz, 1H), 7.48-7.38 (m, 2H), 3.94 (d, J=5.5 Hz, 2H); EI-MS m/z: 358[M]⁺.

### Example 25: Preparation of Compound I-25

Methyl N-((5-(trimethylsilyl)ethynyl)-3-hydroxypicolinoyl)glycinate (200 mg, 0.69 mmol) was dissolved in methanol (10 mL), and then triethylamine (0.2 mL), cuprous iodide (20 mg), TBAF (1 mL), and N-hydroxy-p-fluorobenzimidoyl chloride (142 mg, 0.82 mmol) were added. The mixture was conventionally heated to 50° C. for 4 h and reacted completely. After the reaction was completed, the mixture was filtered under vacuum to remove cuprous iodide and distilled under reduced pressure. The crude product was separated and purified by silica gel column chromatography (petroleum ether:ethyl acetate=2:1) to give a white solid, which was dissolved in tetrahydrofuran (10 mL). A 1 M lithium hydroxide solution (3 mL) was added. The mixture was heated to 30° C. and reacted for 2 h and reacted completely. After the reaction was completed, the mixture was distilled under reduced pressure to remove tetrahydrofuran in the reaction solution. Diluted hydrochloric acid (3 mmol) was added under an ice bath until a white solid was precipitated, and the mixture was filtered under vacuum and dried to give a white product (191 mg, total yield: 81.6%). m.p. 215.1-216.9° C. ¹H NMR (400 MHz, DMSO-d₆) δ 9.40 (s, 1H), 8.72 (d, J=1.8 Hz, 1H), 8.06 (d, J=2.2 Hz, 1H), 8.05 (d, J=3.3 Hz, 2H), 8.03 (d, J=2.2 Hz, 1H), 7.43 (q, J=2.2, 1.5 Hz, 2H), 3.96 (d, J=5.8 Hz, 2H); EI-MS m/z: 358[M]⁺.

### Example 26: Preparation of Compound I-26

Methyl N-((5-(trimethylsilyl)ethynyl)-3-hydroxypicolinoyl)glycinate (200 mg, 0.69 mmol) was dissolved in methanol (10 mL), and then triethylamine (0.2 mL), cuprous iodide (20 mg), TBAF (1 mL), and N-hydroxy-m-difluorobenzimidoyl chloride (157 mg, 0.82 mmol) were added. The mixture was conventionally heated to 50° C. for 4 h and reacted completely. After the reaction was completed, the mixture was filtered under vacuum to remove cuprous iodide and distilled under reduced pressure. The crude product was separated and purified by silica gel column chromatography (petroleum ether:ethyl acetate=2:1) to give a white solid, which was dissolved in tetrahydrofuran (10 mL). A 1 M lithium hydroxide solution (3 mL) was added. The mixture was heated to 30° C. and reacted for 2 h and reacted completely. After the reaction was completed, the mixture was distilled under reduced pressure to remove tetrahydrofuran in the reaction solution. Diluted hydrochloric acid (3 mmol) was added under an ice bath until a white solid was precipitated, and the mixture was filtered under vacuum and dried to give a white product (188 mg, total yield: 76.5%). m.p. 224.6-226.2° C. ¹H NMR (400 MHz, DMSO-d₆) δ 12.66 (s, 1H), 9.43 (s, 1H), 8.67 (s, 1H), 7.98 (s, 1H), 7.86 (s, 1H), 7.63 (d, J=6.8 Hz, 2H), 7.50 (d, J=9.4 Hz, 1H), 3.98 (d, J=5.8 Hz, 2H); EI-MS m/z: 376[M]⁺.

### Example 27: Preparation of Compound I-27

Methyl N-((5-(trimethylsilyl)ethynyl)-3-hydroxypicolinoyl)glycinate (200 mg, 0.69 mmol) was dissolved in methanol (10 mL), and then triethylamine (0.2 mL), cuprous iodide (20 mg), TBAF (1 mL), and N-hydroxy-m-tert-butylbenzimidoyl chloride (173 mg, 0.82 mmol) were added. The mixture was conventionally heated to 50° C. for 4 h and reacted completely. After the reaction was completed, the mixture was filtered under vacuum to remove cuprous iodide and distilled under reduced pressure. The crude product was separated and purified by silica gel column chromatography (petroleum ether:ethyl acetate=2:1) to give a white solid, which was dissolved in tetrahydrofuran (10 mL). A 1 M lithium hydroxide solution (3 mL) was added. The mixture was heated to 30° C. and reacted for 2 h and reacted completely. After the reaction was completed, the mixture was distilled under reduced pressure to remove tetrahydrofuran in the reaction solution. Diluted hydrochloric acid (3 mmol) was added under an ice bath until a white solid was precipitated, and the mixture was filtered under vacuum and dried to give a white product (188 mg, total yield: 76.5%). m.p. 221.1-223.2° C. ¹H NMR (500 MHz, Chloroform-d) δ 8.70 (d, J=1.4 Hz, 1H), 8.36 (t, J=10.3 Hz, 1H), 7.72-7.65 (m, 2H), 7.58 (t, J=1.5 Hz, 1H), 7.53 (t, J=7.5 Hz, 1H), 7.38 (dt, J=7.5, 1.5 Hz, 1H), 7.27 (s, 1H), 4.07 (d, J=10.1 Hz, 2H), 1.37 (s, 7H); EI-MS m/z: 396[M]⁺.

### Example 28: Preparation of Compound I-28

Methyl N-(5-(phenylethynyl)-3-hydroxy-6-methylpicolinoyl)glycinate (214 mg, 0.69 mmol) was dissolved in methanol (10 mL), and then triethylamine (0.2 mL), cuprous iodide (20 mg), TBAF (1 mL), and N-hydroxybenzimidoyl chloride (127 mg, 0.82 mmol) were added. The mixture was conventionally heated to 50° C. for 4 h and reacted completely. After the reaction was completed, the mixture was filtered under vacuum to remove cuprous iodide and distilled under reduced pressure. The crude product was separated and purified by silica gel column chromatography (petroleum ether:ethyl acetate=2:1) to give a white solid, which was dissolved in tetrahydrofuran (10 mL). A 1 M lithium hydroxide solution (3 mL) was added. The mixture was heated to 30° C. and reacted for 2 h and reacted completely. After the reaction was completed, the mixture was distilled under reduced pressure to remove tetrahydrofuran in the reaction solution. Diluted hydrochloric acid (3 mmol) was added under an ice bath until a white solid was precipitated, and the mixture was filtered under vacuum and dried to give a white product (152 mg, total yield: 70.1%). m.p. 278.4-280.3° C. ¹H NMR (500 MHz, Chloroform-d) δ 8.35 (t, J=10.3 Hz, 1H), 7.75-7.67 (m, 3H), 7.52-7.44 (m, 2H), 7.43-7.36 (m, 1H), 7.26 (s, 1H), 4.07 (d, J=10.3 Hz, 2H), 2.79 (s, 2H); EI-MS m/z: 354[M]⁺.

### Example 29: Preparation of Compound I-29

Methyl N-((5-propynyl)-3-hydroxy-6-methylpicolinoyl)glycinate (171 mg, 0.69 mmol) was dissolved in methanol (10 mL), and then triethylamine (0.2 mL), cuprous iodide (20 mg), TBAF (1 mL), and N-hydroxybenzimidoyl chloride (127 mg, 0.82 mmol) were added. The mixture was conventionally heated to 50° C. for 4 h and reacted completely. After the reaction was completed, the mixture was filtered under vacuum to remove cuprous iodide and distilled under reduced pressure. The crude product was separated and purified by silica gel column chromatography (petroleum ether:ethyl acetate=2:1) to give a white solid, which was dissolved in tetrahydrofuran (10 mL). A 1 M lithium hydroxide solution (3 mL) was added. The mixture was heated to 30° C. and reacted for 2 h and reacted completely. After the reaction was completed, the mixture was distilled under reduced pressure to remove tetrahydrofuran in the reaction solution. Diluted hydrochloric acid (3 mmol) was added under an ice bath until a white solid was precipitated, and the mixture was filtered under vacuum and dried to give a white product (100 mg, total yield: 62.3%). m.p. 256.4-258.6° C. ¹H NMR (500 MHz, Chloroform-d) δ 8.63 (d, J=1.4 Hz, 1H), 8.37 (t, J=10.2 Hz, 1H), 7.70 (d, J=1.6 Hz, 1H), 7.62-7.56 (m, 2H), 7.49-7.43 (m, 2H), 7.43-7.36 (m, 1H), 4.07 (d, J=10.3 Hz, 2H), 2.48 (s, 2H); EI-MS m/z: 354[M]⁺.

### Example 30: Preparation of Compound I-30

Methyl N-((5-(3,3-dimethyl-1-butyn)yl)-3-hydroxy-6-methylpicolinoyl)glycinate (200 mg, 0.69 mmol) was dissolved in methanol (10 mL), and then triethylamine (0.2 mL), cuprous iodide (20 mg), TBAF (1 mL), and N-hydroxybenzimidoyl chloride (127 mg, 0.82 mmol) were added. The mixture was conventionally heated to 50° C. for 4 h and reacted completely. After the reaction was completed, the mixture was filtered under vacuum to remove cuprous iodide and distilled under reduced pressure. The crude product was separated and purified by silica gel column chromatography (petroleum ether:ethyl acetate=2:1) to give a white solid, which was dissolved in tetrahydrofuran (10 mL). A 1 M lithium hydroxide solution (3 mL) was added. The mixture was heated to 30° C. and reacted for 2 h and reacted completely. After the reaction was completed, the mixture was distilled under reduced pressure to remove tetrahydrofuran in the reaction solution. Diluted hydrochloric acid (3 mmol) was added under an ice bath until a white solid was precipitated, and the mixture was filtered under vacuum and dried to give a white product (107 mg, total yield: 56.7%). m.p. 288.2-289.9° C. ¹H NMR (500 MHz, Chloroform-d) δ 8.68 (d, J=1.6 Hz, 1H), 8.36 (t, J=10.3 Hz, 1H), 7.75 (d, J=1.6 Hz, 1H), 7.72-7.66 (m, 2H), 7.50-7.43 (m, 2H), 7.46-7.36 (m, 1H), 4.07 (d, J=10.1 Hz, 2H), 1.40 (s, 7H); EI-MS m/z: 396[M]⁺.

### Example 31: Preparation of Compound I-31

Methyl N-((5-(cyclopropyl)ethynyl)-3-hydroxy-6-methylpicolinoyl)glycinate (189 mg, 0.69 mmol) was dissolved in methanol (10 mL), and then triethylamine (0.2 mL), cuprous iodide (20 mg), TBAF (1 mL), and N-hydroxybenzimidoyl chloride (127 mg, 0.82 mmol) were added. The mixture was conventionally heated to 50° C. for 4 h and reacted completely. After the reaction was completed, the mixture was filtered under vacuum to remove cuprous iodide and distilled under reduced pressure. The crude product was separated and purified by silica gel column chromatography (petroleum ether:ethyl acetate=2:1) to give a white solid, which was dissolved in tetrahydrofuran (10 mL). A 1 M lithium hydroxide solution (3 mL) was added. The mixture was heated to 30° C. and reacted for 2 h and reacted completely. After the reaction was completed, the mixture was distilled under reduced pressure to remove tetrahydrofuran in the reaction solution. Diluted hydrochloric acid (3 mmol) was added under an ice bath until a white solid was precipitated, and the mixture was filtered under vacuum and dried to give a white product (76 mg, total yield: 31.2%). m.p. 267.7-269.3° C. ¹H NMR (500 MHz, Chloroform-d) δ 8.67 (d, J=1.6 Hz, 1H), 8.36 (t, J=10.3 Hz, 1H), 7.74-7.66 (m, 3H), 7.50-7.43 (m, 2H), 7.46-7.36 (m, 1H), 4.07 (d, J=10.1 Hz, 2H), 3.51 (p, J=7.0 Hz, 1H), 1.74-1.62 (m, 2H), 0.81-0.69 (m, 2H); EI-MS m/z: 380[M]⁺.

### Example 32: Preparation of Compound I-32

Methyl N-((5-(cyclohexyl)ethynyl)-3-hydroxy-6-methylpicolinoyl)glycinate (218 mg, 0.69 mmol) was dissolved in methanol (10 mL), and then triethylamine (0.2 mL), cuprous iodide (20 mg), TBAF (1 mL), and N-hydroxybenzimidoyl chloride (127 mg, 0.82 mmol) were added. The mixture was conventionally heated to 50° C. for 4 h and reacted completely. After the reaction was completed, the mixture was filtered under vacuum to remove cuprous iodide and distilled under reduced pressure. The crude product was separated and purified by silica gel column chromatography (petroleum ether:ethyl acetate=2:1) to give a white solid, which was dissolved in tetrahydrofuran (10 mL). A 1 M lithium hydroxide solution (3 mL) was added. The mixture was heated to 30° C. and reacted for 2 h and reacted completely. After the reaction was completed, the mixture was distilled under reduced pressure to remove tetrahydrofuran in the reaction solution. Diluted hydrochloric acid (3 mmol) was added under an ice bath until a white solid was precipitated, and the mixture was filtered under vacuum and dried to give a white product (95 mg, total yield: 36.5%). m.p. 270.3-272.5° C. ¹H NMR (500 MHz, Chloroform-d) δ 8.67 (d, J=1.5 Hz, 1H), 8.38 (t, J=10.2 Hz, 1H), 7.74 (d, J=1.5 Hz, 1H), 7.64-7.58 (m, 2H), 7.49-7.42 (m, 2H), 7.42-7.36 (m, 1H), 4.07 (d, J=10.1 Hz, 2H), 3.53 (p, J=6.8 Hz, 1H), 1.81-1.70 (m, 2H), 1.70-1.58 (m, 4H), 1.61-1.36 (m, 4H); EI-MS m/z: 422[M]⁺.

### Example 33: Preparation of Compound I-33

Methyl N-((5-(phenyl)ethynyl)-3-hydroxy-6-methylpicolinoyl)glycinate (214 mg, 0.69 mmol) was dissolved in methanol (10 mL), and then triethylamine (0.2 mL), cuprous iodide (20 mg), TBAF (1 mL), and N-hydroxybenzimidoyl chloride (127 mg, 0.82 mmol) were added. The mixture was conventionally heated to 50° C. for 4 h and reacted completely. After the reaction was completed, the mixture was filtered under vacuum to remove cuprous iodide and distilled under reduced pressure. The crude product was separated and purified by silica gel column chromatography (petroleum ether:ethyl acetate=2:1) to give a white solid, which was dissolved in tetrahydrofuran (10 mL). A 1 M lithium hydroxide solution (3 mL) was added. The mixture was heated to 30° C. and reacted for 2 h and reacted completely. After the reaction was completed, the mixture was distilled under reduced pressure to remove tetrahydrofuran in the reaction solution. Diluted hydrochloric acid (3 mmol) was added under an ice bath until a white solid was precipitated, and the mixture was filtered under vacuum and dried to give a white product (97 mg, total yield: 35.8%). m.p. 289.6-292.3° C. ¹H NMR (500 MHz, Chloroform-d) δ 8.69 (d, J=1.5 Hz, 1H), 8.38 (t, J=10.2 Hz, 1H), 7.77 (d, J=1.5 Hz, 1H), 7.66-7.60 (m, 2H), 7.49-7.37 (m, 8H), 7.37-7.31 (m, 1H), 4.07 (d, J=10.1 Hz, 2H); EI-MS m/z: 416[M]⁺.

### Example 34: Inhibitory Activities of Compounds on FIH—Tested by Fluorescence Polarization (FP Assay)

The ability of the compounds to compete with a fluorophore-labeled HIF-la peptide fragment (FITC-HIF-1α 788-822) for binding to the PHD2 protein was tested using a 384-well black plate (model: Corining #3575) with a final test volume of 60 μL. The compounds tested and FITC-HIF-1α 788-822 were dissolved in DMSO and purified water, respectively, for later use. The compounds were serially diluted in an assay buffer to 12 concentration gradients, and then 20 μL of diluted 300 nM FIH protein was added to each well. Two replicates were set for each compound concentration, and a blank control (20 μL FITC-HIF-1α 788-822+40 μL assay buffer) and a negative control (20 μL FITC-HIF-1α 788-822+20 μL FIH+20 μL assay buffer) were set for each assay. The plate was incubated at room temperature for 1 h and scanned with a Synergy plate reader. The excitation wavelength was set to 485 nm, and the emission wavelength was set to 535 nm. The calculation formula was as follows: % inhibition rate=100−[1−(measured−value blank)/(negative value−blank)], and the inhibition rate corresponding to a specific concentration was obtained. The obtained data were imported into Graphpad prism 8.0 for analysis and fit to obtain IC₅₀ values. The FP test results of the representative compounds are shown in Table 1.

Table 1. Inhibitory activities of some of compounds in the present invention on FIH and related biological activities

| | | Whether or not it can improve |
|---|---|---|
| Compound No. | FIH IC₅₀ (nM) | cellular hyperlipidemia |
| I-1 | 369.2 ± 7.5 | Yes |
| I-2 | 133.3 ± 5.6 | Yes |
| I-3 | 1802 ± 10.2 | Yes |
| I-4 | 432.4 ± 10.4 | Yes |
| I-5 | 247.2 ± 3.2 | Yes |
| I-6 | 233.0 ± 9.2 | Yes |
| I-7 | 648.5 ± 8.6 | Yes |
| I-8 | 906.9 ± 7.4 | Yes |
| I-9 | 943.6 ± 2.2 | Yes |
| I-10 | 336.1 ± 9.5 | Yes |
| I-11 | 487.6 ± 8.7 | Yes |
| I-12 | 1827 ± 6.7 | Yes |
| I-13 | 448.8 ± 5.5 | Yes |
| I-14 | 423.7 ± 7.2 | Yes |
| I-15 | 324.2 ± 9.7 | Yes |
| I-16 | 533.1 ± 3.5 | Yes |
| I-17 | 436.0 ± 9.8 | Yes |
| I-18 | 392.8 ± 3.7 | Yes |
| I-19 | 284.6 ± 1.9 | Yes |
| I-20 | 690.8 ± 1.8 | Yes |
| I-21 | 436.0 ± 1.9 | Yes |
| I-22 | 1354 ± 2.5 | Yes |
| I-23 | 555.3 ± 8.3 | Yes |
| I-24 | 798.8 ± 8.6 | Yes |
| I-25 | 540.0 ± 1.8 | Yes |
| I-26 | 488.7 ± 2.1 | Yes |
| I-27 | 782.4 ± 9.6 | Yes |
| I-28 | 990.8 ± 12.3 | Yes |
| I-29 | 1011 ± 25.1 | Yes |
| I-30 | 1209 ± 22.9 | Yes |
| I-31 | 1920 ± 14.5 | Yes |
| I-32 | 998.2 ± 10.9 | Yes |
| I-33 | 787 ± 9.5 | Yes |
| AKB-6548 | 29031 ± 27 | No |

As can be seen from Table 1, the compounds of the present invention had relatively strong inhibitory activities on FIH, and the IC₅₀ values of 16 compounds were less than 500 nM, wherein the activity of the most active compound reached 100 nM.

In addition, the patent US20070299086A1 discloses a series of proline hydroxylase inhibitors, among which the structure of the compound with a relatively good activity is shown below:

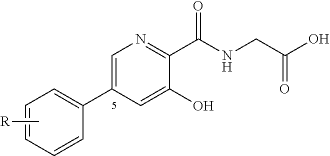

The compounds of the present invention are characterized in that the pyridine parent nucleus, particularly at position 5, contains isoxazole directly linked to the pyridine ring. Compared with the inhibitory activities of the compounds in US20070299086A1 on FIH, it can be found that only the isoxazole compounds of the present invention have relatively good inhibitory activities on FIH under the condition that other groups are substantially the same. The activity comparison results are as follows:

Table 2. Comparison of FIH inhibitory activities between compounds with isoxazole linked to position 5 of pyridine in the present invention and compounds with other structures

| Structure of compound where | | Structure of compound where position 5 | |
|---|---|---|---|
| position 5 of pyridine is linked to | FIH | of pyridine is linked to isoxazole | FIH |
| other structures | (IC₅₀ nM) | and Example No. | (IC₅₀ nM) |
| 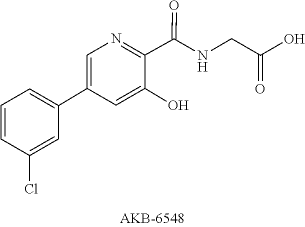 [SMILES: O=C(O)CNC(=O)c1ncc(-c2cccc(Cl)c2)cc1O] | 29031 ± 27 | 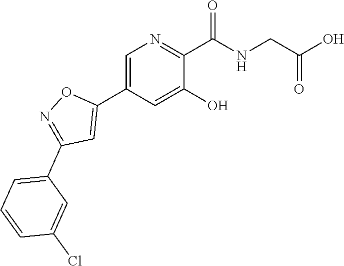 [SMILES: O=C(O)CNC(=O)c1ncc(-c2cc(-c3cccc(Cl)c3)no2)cc1O] Example I-5 | 247.2 ± 3.2 |
| 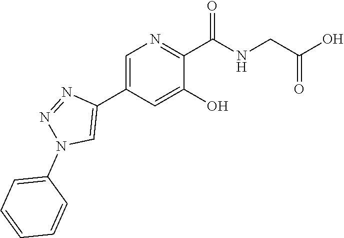 [SMILES: O=C(O)CNC(=O)c1ncc(-c2cn(-c3ccccc3)nn2)cc1O] | 19594 ± 43.8 | 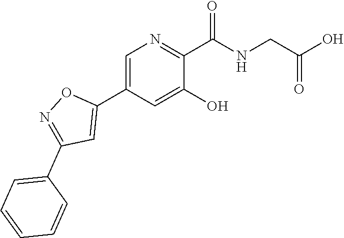 [SMILES: O=C(O)CNC(=O)c1ncc(-c2cc(-c3ccccc3)no2)cc1O] Example I-1 | 369.2 ± 7.5 |
| 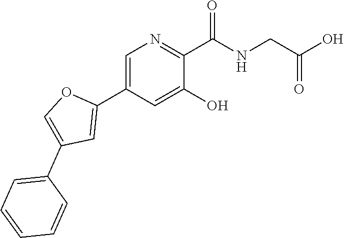 [SMILES: O=C(O)CNC(=O)c1ncc(-c2cc(-c3ccccc3)co2)cc1O] | 9024 ± 36.1 | 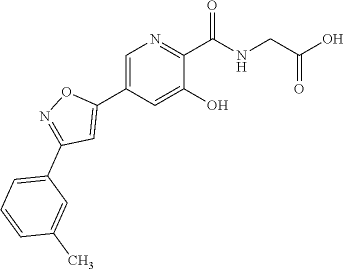 [SMILES: Cc1cccc(-c2cc(-c3cnc(C(=O)NCC(=O)O)c(O)c3)on2)c1] Example I-2 | 133.3 ± 5.6 |
| 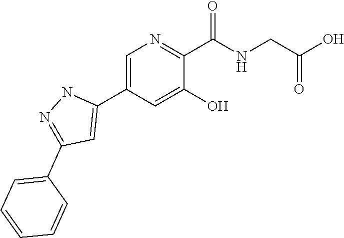 | 22253 ± 56.3 | 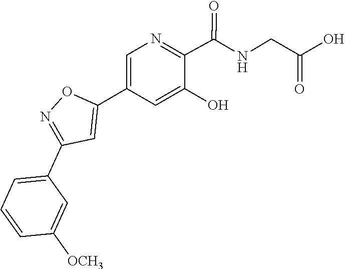 [SMILES: COc1cccc(-c2cc(-c3cnc(C(=O)NCC(=O)O)c(O)c3)on2)c1] Example I-4 | 432.4 ± 10.4 |

As can be seen from the comparison of data of the compounds in Table 2, in the case that other groups were the same, the difference of the aromatic rings linked to position 5 of pyridine could cause significantly different inhibitory activities of the compounds on FIH, and only when position 5 of pyridine was linked to an isoxazole ring, the compounds could show relatively good inhibitory activities on FIH. It means that the linkage of position 5 of pyridine to isoxazole is a necessary condition for ensuring the inhibitory activity on FIH.

### Example 35: Cellular Lipid-Lowering Level Detection

The lipid-lowering ability at the cellular level was confirmed by detecting triglyceride levels of hyperlipidemia cells to determine whether the compounds have the ability to improve hyperlipidemia at the cellular level (J. Med. Chem. 2021, 64(5), 2815-2828). This experiment adopted a human liver cancer cell, HepG2 cell. After a cellular hyperlipidemia model was induced by oleic acid, the cells were incubated and treated with a drug for 24 h. Then, the cells were lysed by ultrasonication, and the content of triglyceride was detected according to the instructions of a triglyceride kit.

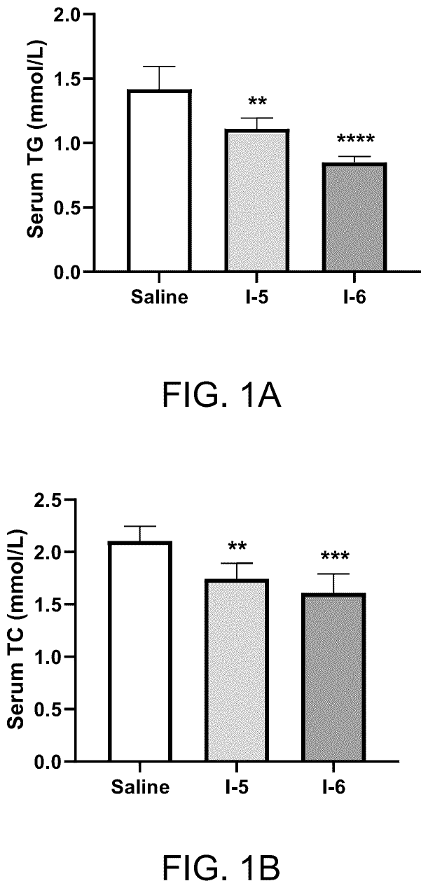

Lipid-lowering tests at the animal level were performed on some of the compounds in Table 1 (dose: 25 mg/kg; model: C57BL/6J mice, male, 7-8 weeks old), and reference was made to J. Med. Chem. 2021, 64 (2), 1037-1053 for the method. As can be seen from FIG. 1A and FIG. 1B, the compounds of the present invention could significantly reduce serum triglyceride and cholesterol at the animal level.

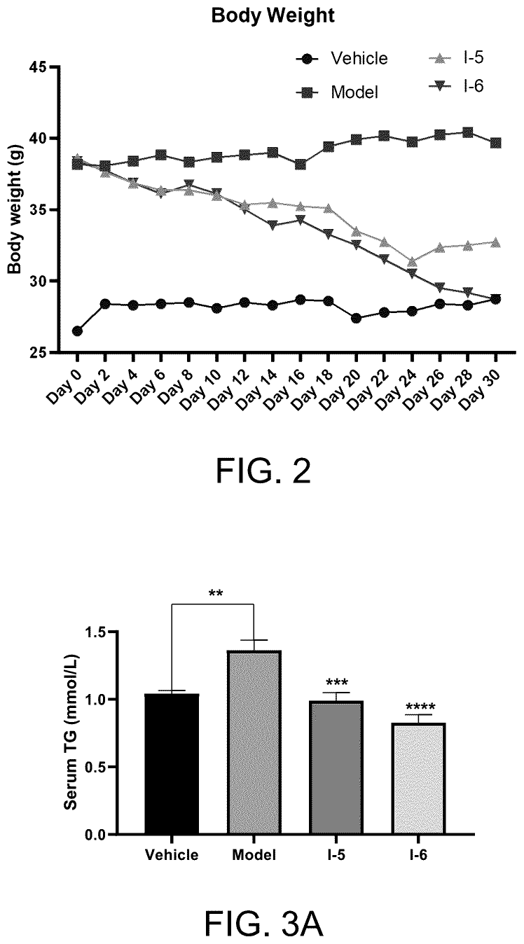

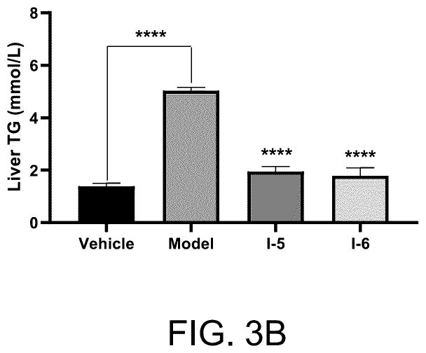

Fatty liver improvement tests at the animal level were performed on some of the compounds in Table 1 (dose: 10 mg/kg; model: C57BL/6J mice, male, 7-8 weeks old), and reference was made to J. Med. Chem. 2021, 64 (2), 1037-1053 for the method. As can be seen from FIG. 2 and FIG. 3A and FIG. 3B, the compounds of the present invention could significantly ameliorate obesity, reduce serum and liver triglycerides, and improve fatty liver at the animal level.

What is claimed is:

1. A 3-hydroxy-5-(isoxazol-5-yl)picolinoyl glycine compound, having a structure of formula (I), wherein the compound comprises a pharmaceutically acceptable salt thereof: 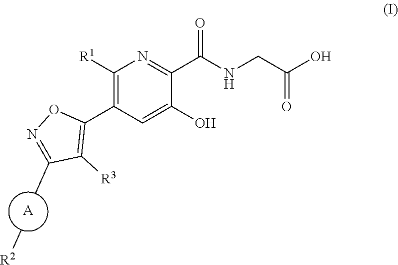
wherein:
A represents an aromatic ring or an aliphatic ring;
R¹ represents hydrogen, halogen, or methyl;
R² represents one or more of hydrogen, C₁-C₄ alkyl, C₁-C₄ alkoxy, C₁-C₄ haloalkyl, halogen, cyano, or phenyl;
R³ represents hydrogen, C₁-C₆ alkyl, C₁-C₆ cycloalkyl, or an aromatic ring.

2. The 3-hydroxy-5-(isoxazol-5-yl)picolinoyl glycine compound according to claim 1, wherein in the structure:
A represents a benzene ring, a naphthalene ring, a 5- to 6-membered aromatic heterocyclic ring, cyclohexane, or cyclopropane.

3. The 3-hydroxy-5-(isoxazol-5-yl)picolinoyl glycine compound according to claim 1, wherein in the structure:
R² represents one or more of hydrogen, methyl, tert-butyl, methoxy, trifluoromethyl, fluoro, chloro, bromo, cyano, or phenyl.

4. The 3-hydroxy-5-(isoxazol-5-yl)picolinoyl glycine compound according to claim 1, wherein in the structure:
R³ represents hydrogen, methyl, tert-butyl, cyclopropyl, cyclohexyl, or phenyl.

5. The 3-hydroxy-5-(isoxazol-5-yl)picolinoyl glycine compound according to claim 1, wherein the compound is selected from any one of the following compounds:

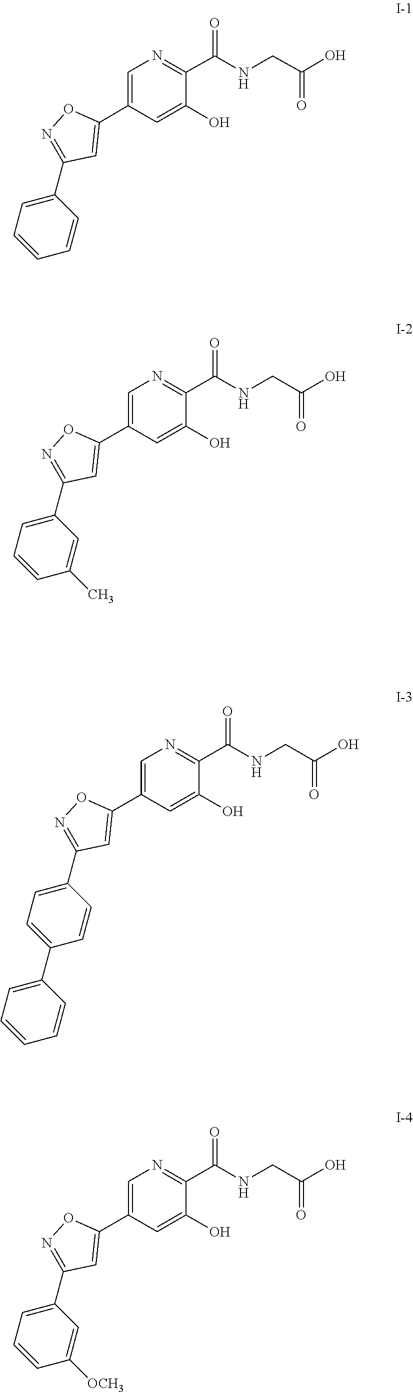 [SMILES: O=C(O)CNC(=O)c1ncc(-c2cc(-c3ccccc3)no2)cc1O ; Cc1cccc(-c2cc(-c3cnc(C(=O)NCC(=O)O)c(O)c3)on2)c1 ; O=C(O)CNC(=O)c1ncc(-c2cc(-c3ccc(-c4ccccc4)cc3)no2)cc1O ; COc1cccc(-c2cc(-c3cnc(C(=O)NCC(=O)O)c(O)c3)on2)c1]

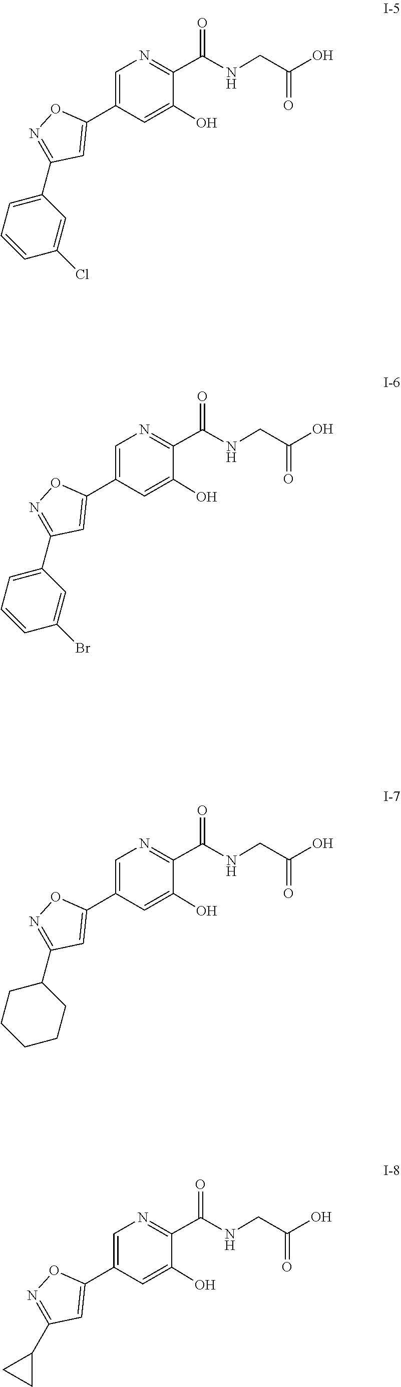 [SMILES: O=C(O)CNC(=O)c1ncc(-c2cc(-c3cccc(Cl)c3)no2)cc1O ; O=C(O)CNC(=O)c1ncc(-c2cc(-c3cccc(Br)c3)no2)cc1O ; O=C(O)CNC(=O)c1ncc(-c2cc(C3CCCCC3)no2)cc1O ; O=C(O)CNC(=O)c1ncc(-c2cc(C3CC3)no2)cc1O]

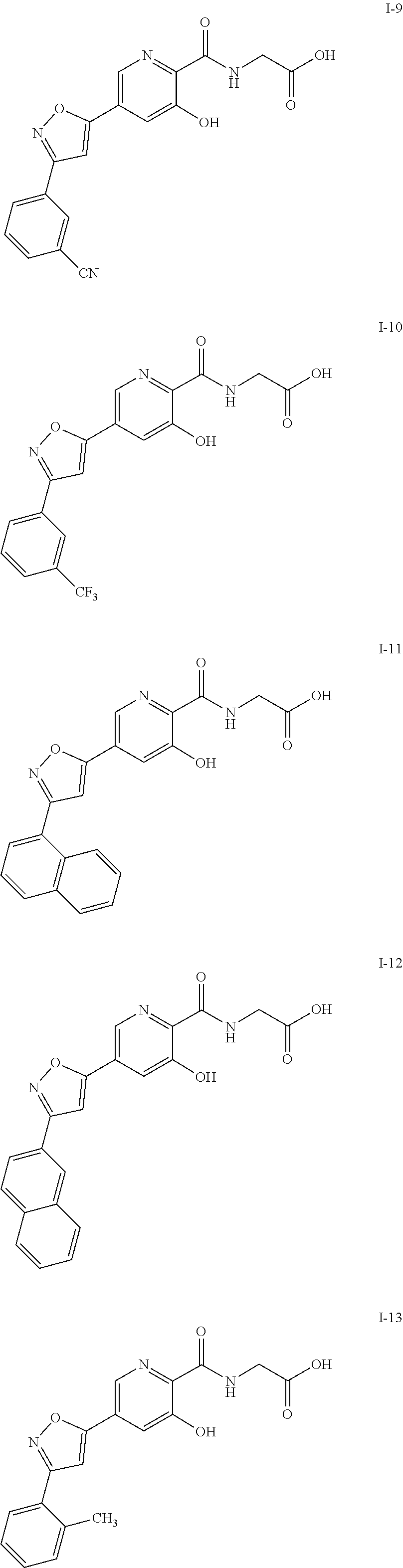 [SMILES: N#Cc1cccc(-c2cc(-c3cnc(C(=O)NCC(=O)O)c(O)c3)on2)c1 ; O=C(O)CNC(=O)c1ncc(-c2cc(-c3cccc(C(F)(F)F)c3)no2)cc1O ; O=C(O)CNC(=O)c1ncc(-c2cc(-c3cccc4ccccc34)no2)cc1O ; O=C(O)CNC(=O)c1ncc(-c2cc(-c3ccc4ccccc4c3)no2)cc1O ; Cc1ccccc1-c1cc(-c2cnc(C(=O)NCC(=O)O)c(O)c2)on1]

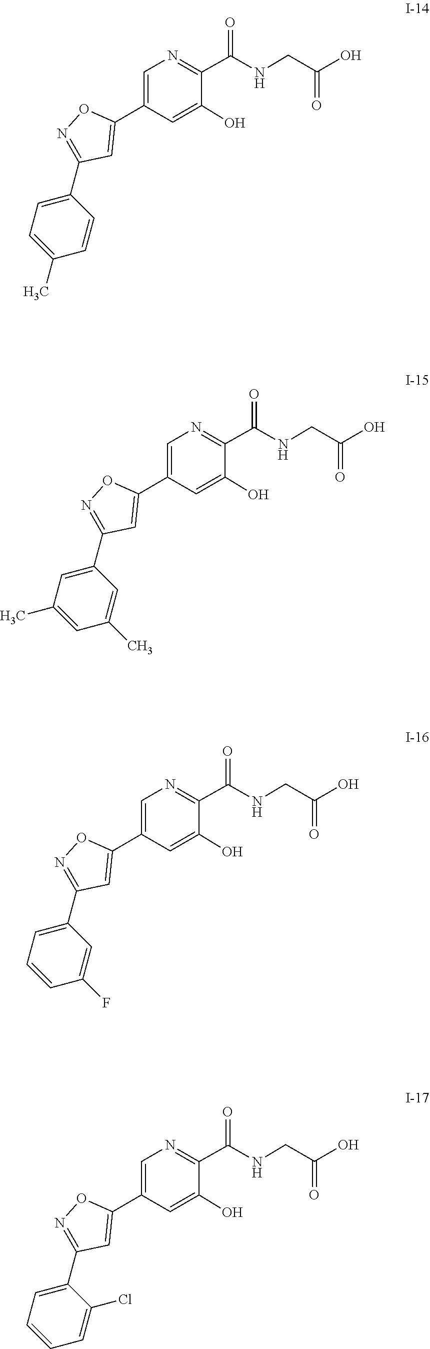 [SMILES: Cc1ccc(-c2cc(-c3cnc(C(=O)NCC(=O)O)c(O)c3)on2)cc1 ; Cc1cc(C)cc(-c2cc(-c3cnc(C(=O)NCC(=O)O)c(O)c3)on2)c1 ; O=C(O)CNC(=O)c1ncc(-c2cc(-c3cccc(F)c3)no2)cc1O ; O=C(O)CNC(=O)c1ncc(-c2cc(-c3ccccc3Cl)no2)cc1O]

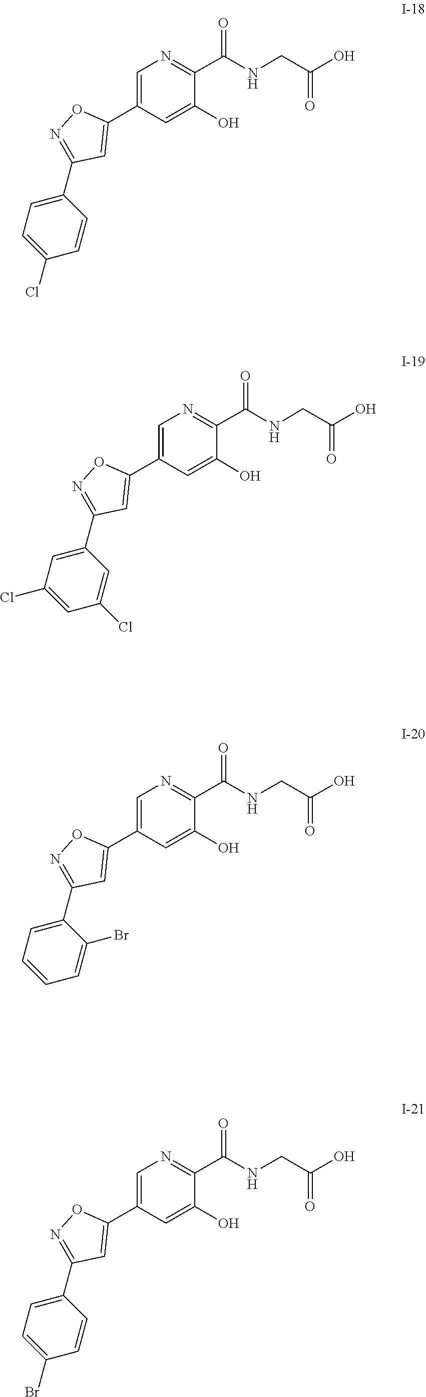 [SMILES: O=C(O)CNC(=O)c1ncc(-c2cc(-c3ccc(Cl)cc3)no2)cc1O ; O=C(O)CNC(=O)c1ncc(-c2cc(-c3cc(Cl)cc(Cl)c3)no2)cc1O ; O=C(O)CNC(=O)c1ncc(-c2cc(-c3ccccc3Br)no2)cc1O ; O=C(O)CNC(=O)c1ncc(-c2cc(-c3ccc(Br)cc3)no2)cc1O]

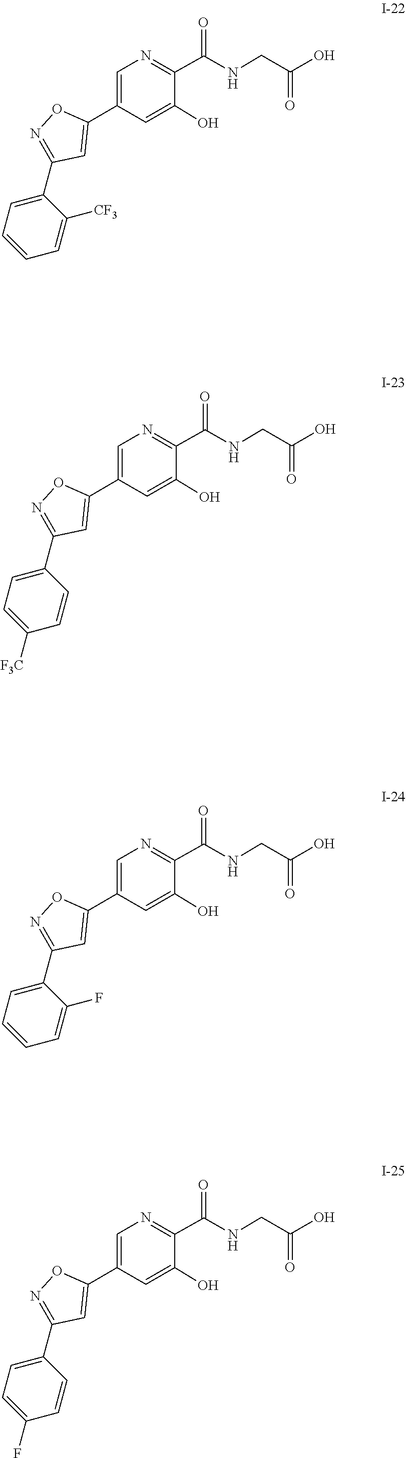 [SMILES: O=C(O)CNC(=O)c1ncc(-c2cc(-c3ccccc3C(F)(F)F)no2)cc1O ; O=C(O)CNC(=O)c1ncc(-c2cc(-c3ccc(C(F)(F)F)cc3)no2)cc1O ; O=C(O)CNC(=O)c1ncc(-c2cc(-c3ccccc3F)no2)cc1O ; O=C(O)CNC(=O)c1ncc(-c2cc(-c3ccc(F)cc3)no2)cc1O]

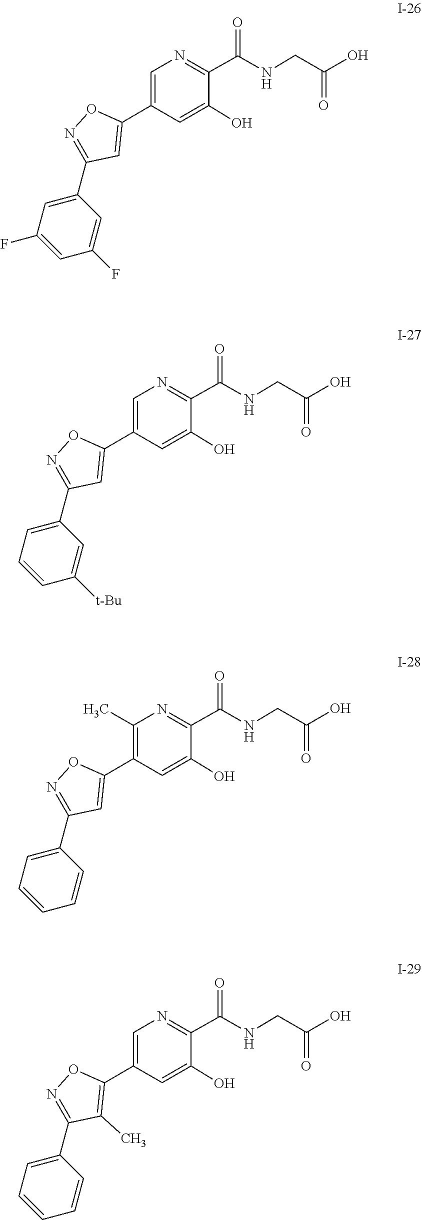 [SMILES: O=C(O)CNC(=O)c1ncc(-c2cc(-c3cc(F)cc(F)c3)no2)cc1O ; CC(C)(C)c1cccc(-c2cc(-c3cnc(C(=O)NCC(=O)O)c(O)c3)on2)c1 ; Cc1nc(C(=O)NCC(=O)O)c(O)cc1-c1cc(-c2ccccc2)no1 ; Cc1c(-c2ccccc2)noc1-c1cnc(C(=O)NCC(=O)O)c(O)c1]

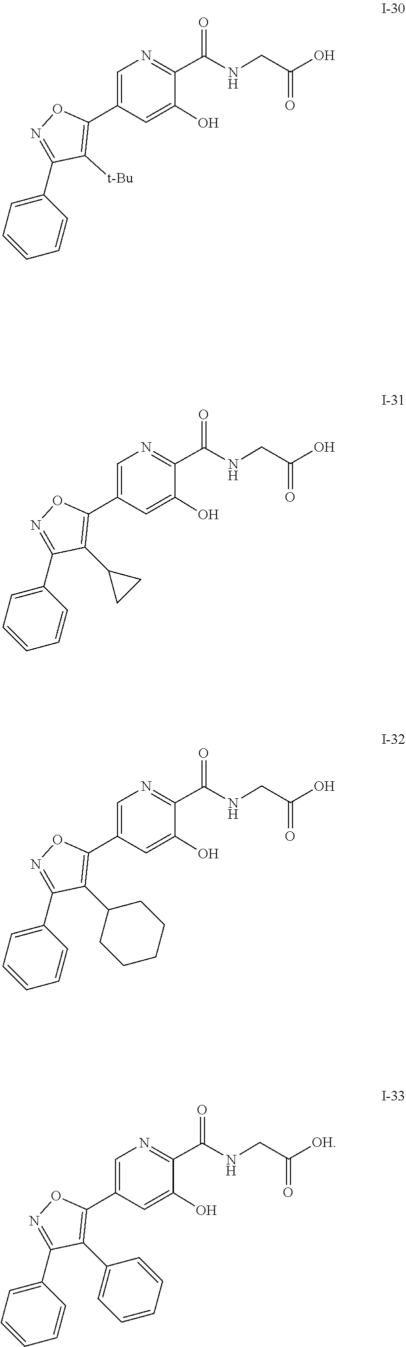 [SMILES: CC(C)(C)c1c(-c2ccccc2)noc1-c1cnc(C(=O)NCC(=O)O)c(O)c1 ; O=C(O)CNC(=O)c1ncc(-c2onc(-c3ccccc3)c2C2CC2)cc1O ; O=C(O)CNC(=O)c1ncc(-c2onc(-c3ccccc3)c2C2CCCCC2)cc1O ; O=C(O)CNC(=O)c1ncc(-c2onc(-c3ccccc3)c2-c2ccccc2)cc1O]

6. The 3-hydroxy-5-(isoxazol-5-yl)picolinoyl glycine compound according to claim 1, wherein the pharmaceutically acceptable salt is a salt formed by the compound and an acid or a base; the acid is hydrochloric acid, hydrobromic acid, carbonic acid, sulfuric acid, phosphoric acid, methanesulfonic acid, benzenesulfonic acid, p-toluenesulfonic acid, naphthalenesulfonic acid, citric acid, malic acid, tartaric acid, lactic acid, pyruvic acid, acetic acid, maleic acid, succinic acid, fumaric acid, salicylic acid, phenylacetic acid, or mandelic acid; the base is an inorganic base comprising an alkali metal cation, an alkaline earth metal cation, or an ammonium cation salt, or an organic amine.

7. A preparation method for the 3-hydroxy-5-(isoxazol-5-yl)picolinoyl glycine compound according to claim 1, wherein the preparation method is as follows:
compound (III) is subjected to a cyclization reaction and a hydrolysis reaction to give compound (I); 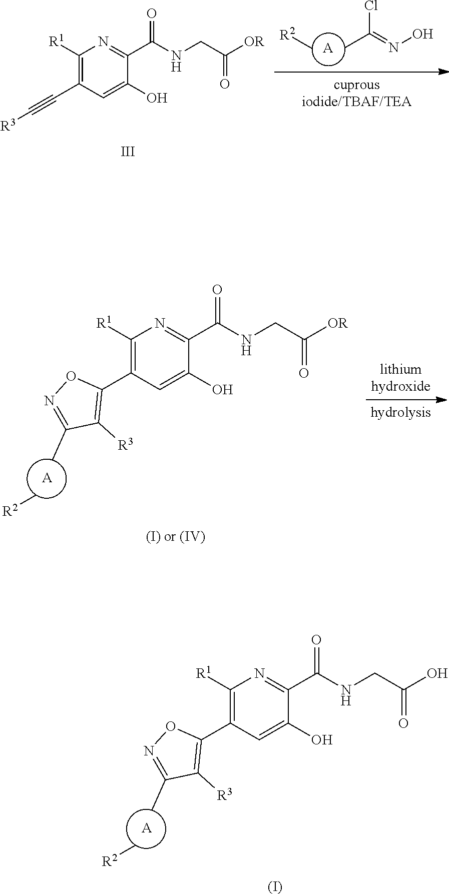
wherein A, R¹, R², and R³ are as defined in claim 1; R represents hydrogen, C₁-C₄ aliphatic hydrocarbyl or benzyl;
a corresponding acid or base is subjected to a salt-forming reaction with compound (I) prepared by the above method to give the pharmaceutically acceptable salt of the compound.

8. A pharmaceutical composition comprising the 3-hydroxy-5-(isoxazol-5-yl)picolinoyl glycine compound according to claim 1, and a pharmaceutically acceptable carrier.

9. Use of the 3-hydroxy-5-(isoxazol-5-yl)picolinoyl glycine compound according to claim 1 or a pharmaceutical composition in the preparation of an inhibitor drug for a factor inhibiting HIF,
wherein the pharmaceutical composition comprises the 3-hydroxy-5-(isoxazol-5-yl)picolinoyl glycine compound according to claim 1, and a pharmaceutically acceptable carrier.

10. The use according to claim 9, wherein the drug is used for treating a lipid metabolic disease.

11. The 3-hydroxy-5-(isoxazol-5-yl)picolinoyl glycine compound according to claim 2, wherein the pharmaceutically acceptable salt is a salt formed by the compound and an acid or a base; the acid is hydrochloric acid, hydrobromic acid, carbonic acid, sulfuric acid, phosphoric acid, methanesulfonic acid, benzenesulfonic acid, p-toluenesulfonic acid, naphthalenesulfonic acid, citric acid, malic acid, tartaric acid, lactic acid, pyruvic acid, acetic acid, maleic acid, succinic acid, fumaric acid, salicylic acid, phenylacetic acid, or mandelic acid; the base is an inorganic base comprising an alkali metal cation, an alkaline earth metal cation, or an ammonium cation salt, or an organic amine.

12. The 3-hydroxy-5-(isoxazol-5-yl)picolinoyl glycine compound according to claim 3, wherein the pharmaceutically acceptable salt is a salt formed by the compound and an acid or a base; the acid is hydrochloric acid, hydrobromic acid, carbonic acid, sulfuric acid, phosphoric acid, methanesulfonic acid, benzenesulfonic acid, p-toluenesulfonic acid, naphthalenesulfonic acid, citric acid, malic acid, tartaric acid, lactic acid, pyruvic acid, acetic acid, maleic acid, succinic acid, fumaric acid, salicylic acid, phenylacetic acid, or mandelic acid; the base is an inorganic base comprising an alkali metal cation, an alkaline earth metal cation, or an ammonium cation salt, or an organic amine.

13. The 3-hydroxy-5-(isoxazol-5-yl)picolinoyl glycine compound according to claim 4, wherein the pharmaceutically acceptable salt is a salt formed by the compound and an acid or a base; the acid is hydrochloric acid, hydrobromic acid, carbonic acid, sulfuric acid, phosphoric acid, methanesulfonic acid, benzenesulfonic acid, p-toluenesulfonic acid, naphthalenesulfonic acid, citric acid, malic acid, tartaric acid, lactic acid, pyruvic acid, acetic acid, maleic acid, succinic acid, fumaric acid, salicylic acid, phenylacetic acid, or mandelic acid; the base is an inorganic base comprising an alkali metal cation, an alkaline earth metal cation, or an ammonium cation salt, or an organic amine.

14. The 3-hydroxy-5-(isoxazol-5-yl)picolinoyl glycine compound according to claim 5, wherein the pharmaceutically acceptable salt is a salt formed by the compound and an acid or a base; the acid is hydrochloric acid, hydrobromic acid, carbonic acid, sulfuric acid, phosphoric acid, methanesulfonic acid, benzenesulfonic acid, p-toluenesulfonic acid, naphthalenesulfonic acid, citric acid, malic acid, tartaric acid, lactic acid, pyruvic acid, acetic acid, maleic acid, succinic acid, fumaric acid, salicylic acid, phenylacetic acid, or mandelic acid; the base is an inorganic base comprising an alkali metal cation, an alkaline earth metal cation, or an ammonium cation salt, or an organic amine.

15. A preparation method for the 3-hydroxy-5-(isoxazol-5-yl)picolinoyl glycine compound according to claim 2,
wherein the preparation method is as follows:
compound (III) is subjected to a cyclization reaction and a hydrolysis reaction to give compound (I); 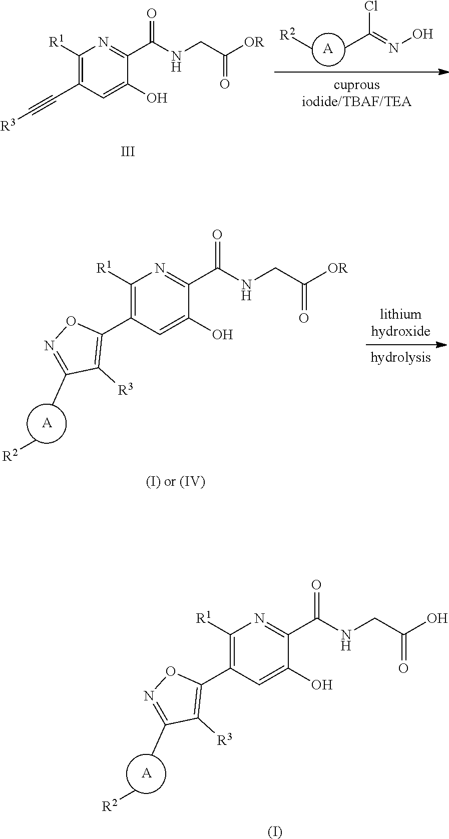
wherein A, R¹, R², and R³ are as defined in claim 2; R represents hydrogen, C₁-C₄ aliphatic hydrocarbyl or benzyl;
a corresponding acid or base is subjected to a salt-forming reaction with compound (I) prepared by the above method to give the pharmaceutically acceptable salt of the compound.

16. A preparation method for the 3-hydroxy-5-(isoxazol-5-yl)picolinoyl glycine compound according to claim 3,
wherein the preparation method is as follows:
compound (III) is subjected to a cyclization reaction and a hydrolysis reaction to give compound (I); 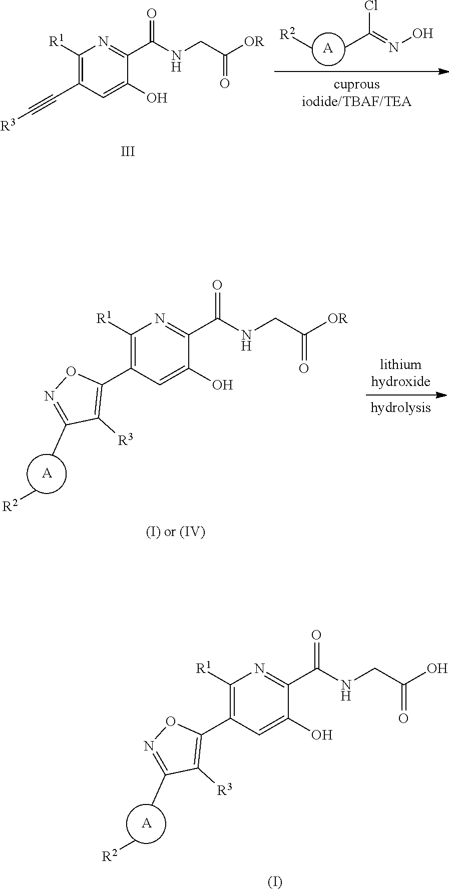
wherein A, R¹, R², and R³ are as defined in claim 3; R represents hydrogen, C₁-C₄ aliphatic hydrocarbyl or benzyl;
a corresponding acid or base is subjected to a salt-forming reaction with compound (I) prepared by the above method to give the pharmaceutically acceptable salt of the compound.

17. A preparation method for the 3-hydroxy-5-(isoxazol-5-yl)picolinoyl glycine compound according to claim 4,
wherein the preparation method is as follows:
compound (III) is subjected to a cyclization reaction and a hydrolysis reaction to give compound (I); 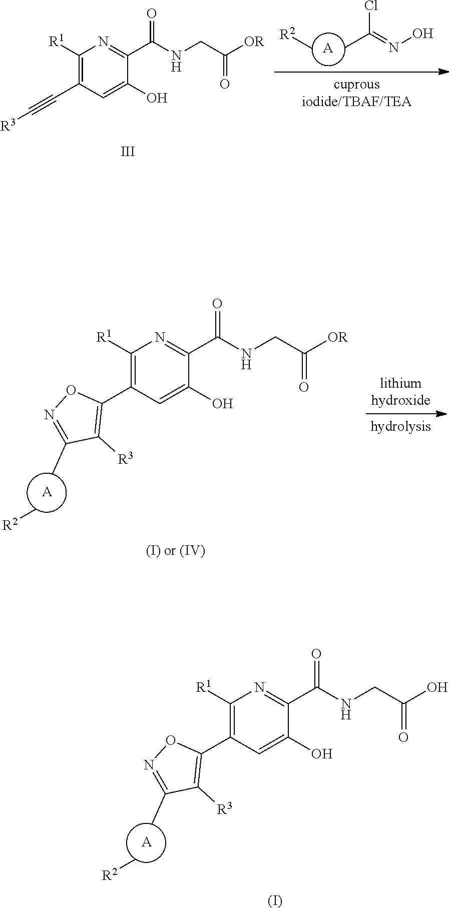
wherein A, R¹, R², and R³ are as defined in claim 4; R represents hydrogen, C₁-C₄ aliphatic hydrocarbyl or benzyl;
a corresponding acid or base is subjected to a salt-forming reaction with compound (I) prepared by the above method to give the pharmaceutically acceptable salt of the compound.

18. A preparation method for the 3-hydroxy-5-(isoxazol-5-yl)picolinoyl glycine compound according to claim 5,
wherein the preparation method is as follows:
compound (III) is subjected to a cyclization reaction and a hydrolysis reaction to give compound (I); 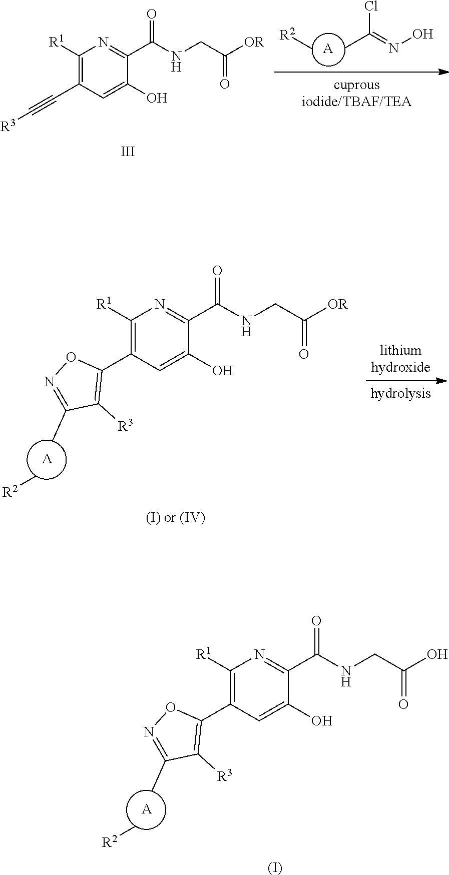
wherein A, R¹, R², and R³ are as defined in claim 5; R represents hydrogen, C₁-C₄ aliphatic hydrocarbyl or benzyl;
a corresponding acid or base is subjected to a salt-forming reaction with compound (I) prepared by the above method to give the pharmaceutically acceptable salt of the compound.

19. A pharmaceutical composition comprising the 3-hydroxy-5-(isoxazol-5-yl)picolinoyl glycine compound according to claim 2, and a pharmaceutically acceptable carrier.

20. A pharmaceutical composition comprising the 3-hydroxy-5-(isoxazol-5-yl)picolinoyl glycine compound according to claim 3, and a pharmaceutically acceptable carrier.
